(19) United States
(12) Patent Application Publication
(10) Pub. No.: US 2025/0270536 A1
(43) Pub. Date: Aug. 28, 2025

(54) BACTERIAL COMPOSITIONS AND METHODS FOR GROWING BACTERIA ON PARTICLES

(71) Applicant: MYBIOTICS PHARMA LTD., Rehovot (IL)

(72) Inventors: David DABOUSH, Mishmar David (IL); Lior WEISSMAN, Ness-Ziona (IL); Igor LEV, Frankfurt am Main (DE); Stanislav MASHYN, Rehovot (IL); Sergey BORODANOV, Rehovot (IL)

(21) Appl. No.: 18/853,275

(22) PCT Filed: Apr. 4, 2023
(86) PCT No.: PCT/IL2023/050372

§ 371 Date: Oct. 1, 2024

Related U.S. Application Data
(60) Provisional application No. 63/327,455, filed on Apr. 5, 2022.

(51) Int. Cl. C12N 11/14; C12N 1/20

(52) U.S. Cl. CPC C12N 11/14; C12N 1/20

(57) ABSTRACT

The present invention is directed, inter alia, to compositions comprising a population of at least one strain of bacteria at least partially attached to particles, the particles comprise (i) a polysaccharide-based material; and (ii) a metal carbonate. Uses of the compositions, e.g., for prevention or treatment of dysbiosis, and methods for the preparation thereof are further provided herein.

### CROSS REFERENCE TO RELATED APPLICATIONS

This application claims the benefit of priority of U.S. Provisional Patent Application No. 63/327,455, filed on Apr. 5, 2022, titled “COMPOSITIONS AND METHODS FOR GROWING BACTERIA ON PARTICLES”, the contents of which are incorporated herein by reference in their entirety.

### FIELD OF THE INVENTION

The present invention is in the field of microbiology, and particularly relates to bacterial compositions in attached and/or aggregated form, and methods of preparing and using same.

### BACKGROUND

The human body contains an abundant and diverse microbial community, creating a network of bacterial-human cell interactions that greatly affects our health status and even our behavioral patterns.

There is a growing body of evidence advocating the use of probiotics to promote human health, e.g., benefiting the immune system, suppressing infections, etc.

A wide variety of bacterial compositions have been developed for preventing or treating various diseases including chronic disorders as dysbiosis, obesity, infections, colitis, inflammatory bowel disease (such as Crohn's disease), autoimmune diseases, cancer, poor vaginal health, and various skin conditions.

However, due to harsh environmental conditions during passage to and at the site of interest, one of the challenges in biotherapeutics delivery, is the successful survival, colonization, and expansion of the delivered bacteria.

Several attempts have been made to develop bacterial compositions that are better suited for achieving bacterial colonization and providing a lasting beneficial effect. Each of the known compositions and methods have some advantages and disadvantages.

There remains an unmet need for alternative compositions, e.g., compositions comprising bacteria attached to alternative particles, that can overcome the disadvantages of existing compositions and provide advantageous methods for preventing or treating diseases and/or conditions.

Advantageously, such alternative particles can be at least partially dissolved during the preparation of the composition while maintaining the bacteria's viability and beneficial effect, thereby enabling production of a composition comprising a decreased content of inert material and an increased relative content of the bacteria in the final product.

### SUMMARY

In some aspects of the invention, there is provided a composition comprising a population of at least one strain of bacteria at least partially attached to particles, wherein the particles comprise: (i) a polysaccharide-based material; and (ii) a metal carbonate.

In another aspect of the invention, there is provided a method for preparing a composition comprising a population of at least one strain of bacteria at least partially attached to particles, wherein the particles comprise: (i) a polysaccharide-based material; and (ii) a metal carbonate, the method comprising the steps of: providing a population of at least one strain of bacteria; providing particles comprising: (i) a polysaccharide-based material; and (ii) a metal carbonate; contacting the population of at least one strain of bacteria with the particles, wherein the contacting is carried out in a solution; and allowing the population of at least one strain of bacteria to at least partially attach to the particles; thereby preparing the composition comprising the population of at least one strain of bacteria at least partially attached to the particles.

In some embodiments, the particles are water-insoluble particles.

In some embodiments, the metal carbonate is not being produced by the at least one strain of bacteria.

In some embodiments, the composition having at least one characteristic selected from: (a) comprises the particles in a form of a composite comprising the polysaccharide-based material and the metal carbonate; and (b) comprises each of the polysaccharide-based material and the metal carbonate within a different or separated particle.

In some embodiments, the particles have at least one characteristic selected from: (a) are in a form of a composite comprising the polysaccharide-based material and the metal carbonate; and (b) comprise each of the polysaccharide-based material and the metal carbonate within separated particles.

In some embodiments, at least a portion of the particles are in a form of a composite comprising: (i) the polysaccharide-based material; and (ii) the metal carbonate. In some embodiments, a portion of the particles comprise the polysaccharide-based material and the metal carbonate not-blended together within the same particle.

In some embodiments, each particle may comprise additional material(s). In some embodiments, the particles comprise at least one additional material.

In some embodiments, the additional material is or comprises dicalcium phosphate (DCP).

In some embodiments, the particles further comprise dicalcium phosphate (DCP).

In some embodiments, the composition and/or method further comprise particles being devoid of polysaccharide-based material and metal carbonate.

In some embodiments, the at least one strain of bacteria is attached to at least one of: a surface of the polysaccharide-based material; and a surface of the metal carbonate.

In some embodiments, the particles comprise a plurality of types of polysaccharide-based materials. In some embodiments, the particles comprise a plurality of types of metal carbonate.

In some embodiments, the polysaccharide-based material is or comprises a cellulose derivative.

In some embodiments, the polysaccharide-based material is or comprises microcrystalline cellulose (MCC).

In some embodiments, the metal carbonate is or comprises calcium carbonate.

In some embodiments, the composition comprises the metal carbonate at a concentration of at most 85% (w/w) out of the total weight of the particles within the composition, based on the dry weight of the particles.

In some embodiments, the composition characterized by anyone of: (i) comprises the metal carbonate at a concentration of at least 15% (w/w) out of the total weight of the particles within the composition, based on the dry weight of the particles; or (ii) further comprising metal phosphate comprising particles, and wherein the metal phosphate, and the metal carbonate constitute at least 15% (w/w) out of the total weight of the particles within the composition, based on the dry weight of the particles.

In some embodiments, the polysaccharide-based material and the metal carbonate are at a w/w ratio of between 1:10 and 10:1, based on the dry weight of the particles. In some embodiments, the polysaccharide-based material and the metal carbonate are at a w/w ratio of between 1:6 and 6:1, based on the dry weight of the particles.

In some embodiments, the composition comprises a trace amount of metal carbonate.

In some embodiments, the composition further comprises bacteria un-attached to the particles. In some embodiments, the composition further comprises planktonic bacteria.

In some embodiments, the composition comprises two or more strains of bacteria.

In some embodiments, the composition comprises a trace amount of the metal carbonate.

In some embodiments, the composition is provided in solid form.

In some embodiments, the composition further comprising a pharmaceutically acceptable carrier or excipient.

In some embodiments, the composition is obtained by a process comprising the steps: contacting, mixing, incubating and/or culturing the population of at least one strain of bacteria with the particles, and allowing the bacteria to at least partially attach to the particles.

In some embodiments, the composition is obtained by a process comprising a step of: culturing the population of at least one strain of bacteria in the presence of particles.

In some embodiments, the invention provides a method for preparing a composition as disclosed herein.

In some embodiments, allowing the population of at least one strain of bacteria to at least partially attach to the particles comprises culturing the population with the particles.

In some embodiments, allowing the population of at least one strain of bacteria to at least partially attach to the particles comprises culturing the population with the particles for a time sufficient for the population to attach to the particles such as for 1 hour to 15 days.

In some embodiments, allowing the population of at least one strain of bacteria to at least partially attach to the particles comprises culturing the population with the particles until the population attaches the particles such as for 1 hour to 15 days.

In some embodiments, the method further comprises a step of mixing the population of at least one strain of bacteria with the particles in dry form prior to the contacting step.

In some embodiments, contacting comprises mixing, incubating and/or culturing. In some embodiments, inoculating is the initial stage of the culturing step and is carried out in the presence of the particles. In some embodiments, contacting is carried out in a solution selected from the group consisting of: saline, phosphate buffered saline, and a growth medium. In some embodiments, the solution is a growth medium. In some embodiments, the different steps of the method can be carried out in the same or in a different solution. In some embodiments, the contacting step comprises culturing the population of at least one strain of bacteria in a growth medium comprising the particles.

In some embodiments, the method comprises contacting two or more strains of bacteria with the particles.

In some embodiments, at least a portion of the particles used in the method according to the invention form of a composite comprising the polysaccharide-based material; and the metal carbonate. In some embodiments, a portion of the particles used in the method according to the invention comprise the polysaccharide-based material and the metal carbonate not-blended together within the same particle.

In some embodiments, during the contacting step, the particles have at least one characteristic selected from: (a) are in a form of a composite comprising the polysaccharide-based material and the metal carbonate; and (b) comprise each of the polysaccharide-based material and the metal carbonate within a different or separated particle.

In some embodiments, the provided particles comprise at least one additional material.

In some embodiments, the weight of the metal carbonate with respect to the volume of the solution is in a range of between 9 g per 1 L and 215 g per 1 L, based on the dry weight of the particles.

In some embodiments, the total weight of the provided particles with respect to the volume of the solution is in a range of between 60 g per 1 L and 250 g per 1 L, based on the dry weight of the particles.

In some embodiments, the provided particles comprise the metal carbonate at a concentration of at most 85% (w/w) out of the total weight of the particles, based on the dry weight of the particles.

In some embodiments, the provided particles are characterized by anyone of: (i) comprise the metal carbonate at a concentration of at least 15% (w/w) out of the total weight of the particles, based on the dry weight of the particles; or (ii) further comprise metal phosphate, and wherein the metal phosphate, and the metal carbonate constitute at least 15% out of the total weight of the particles, based on the dry weight of the particles.

In some embodiments, the method further comprises a step of eliminating the metal carbonate from the particles by adding an acid, CO₂, or both.

In another aspect of the invention, a method for treating a patient is disclosed. The method comprises: administering the composition according to the invention to a patient.

According to another aspect, there is provided a method for preventing or treating dysbiosis in a subject in need, comprising administering to the subject an effective amount of the composition according to the invention.

In some embodiments, the invention provides a method for preparing a composition as disclosed herein, the method comprising the steps of: inoculating a population comprising at least one strain of bacteria in a solution; and culturing the population comprising the at least one strain of bacteria in a solution comprising particles for a time sufficient for the bacteria to at least partially attach to the particles, wherein the particles comprise: (i) a cellulose-based material; and (ii) a metal carbonate.

According to another aspect, there is provided a composition produced according to the method of the invention, as disclosed herein.

According to another aspect, there is provided a pharmaceutical composition comprising the composition of the invention, as disclosed herein, and an acceptable carrier.

In some embodiments, the pharmaceutical composition is for use in the treatment or prevention of dysbiosis in a subject in need thereof.

In some embodiments, the composition further comprises an acceptable carrier. In some embodiments, the composition is for use in the treatment or prevention of dysbiosis in a subject in need thereof. In some embodiments, the composition is for agricultural or veterinary use.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a bar graph showing the bacterial count of Bifidobacterium bifidum (Log CFU/ml) following culturing in planktonic growth form (in the absence of particles); in the presence of particles for a combined particle-attached and non-attached bacterial fractions (marked as “Group 1”); only in the particle-attached bacterial fraction (marked as “Group 2”); and following partial elimination of the metal carbonate particles from the particle-attached bacterial fraction, i.e., elimination of the metal carbonate particles of Group 2 compositions, by acid (marked as “Group 3”).

FIG. 2 includes a bar graph showing the survival rate of a wet (at the end of culturing) Lactobacillus plantarum culture following exposure to increasing H₂O₂ concentrations. The resistance of the attached bacterial fraction prepared by different culturing methods were tested: (i) culturing according to an embodiment of the invention in the presence of CaCO₃:MCC particle mix (1:1 w/w ratio); and (ii) bacteria cultured on a 96 w/p. Planktonic growth form was used as a reference. Following the H₂O₂ exposure, the total bacterial count (CFU) was measured. The results are presented in percentages as compared to the control un-treated bacteria of the respective group (‘no addition of H₂O₂’ considered as 100%).

FIG. 3 includes a bar graph showing the survival of lyophilized Lactobacillus plantarum culture following reconstitution and exposure to increasing H₂O₂ concentrations. The resistance was tested in the attached bacterial fraction prepared by culturing according to an embodiment of the invention in the presence of CaCO₃:MCC particle mix (1:1 w/w ratio); and in planktonic growth form. Following the H₂O₂ stress, the total bacterial count (CFU) was measured. The results are presented in percentages as compared to the control un-treated bacteria of the respective group (‘no addition of H₂O₂’ considered as 100%).

### DETAILED DESCRIPTION

According to some embodiments, there is provided a composition comprising a population comprising at least one strain of bacteria at least partially attached to particles.

According to some embodiments, there is provided a composition comprising a population comprising at least one strain of bacteria grown in the presence of particles as detailed herein.

According to some embodiments, there is provided a method for producing a composition comprising a population comprising at least one strain of bacteria at least partially attached to particles.

According to some embodiments, there is provided a method for obtaining an increased attachment of bacteria to particles, e.g., when using particles comprising: (i) a polysaccharide-based material; and (ii) a metal carbonate, as compared to using particles comprising a polysaccharide-based material in the absence of metal carbonate particles.

According to some embodiments, there is provided a method for growing a population comprising at least one strain of bacteria (e.g., gut-derived bacteria, skin-derived bacteria, vaginal-derived bacteria) comprising a step of admixing, contacting and/or culturing in the presence of particles as detailed herein.

In some embodiments, the particles are added, mixed, contacted (e.g., incubated) and/or cultured with the population of at least one strain of bacteria in a solution such as a saline solution, phosphate buffer solution, a growth medium and the like. In some embodiments, the population of at least one strain of bacteria and the particles are contacted when the bacteria and/or the particles are in a wet or hydrated form. In some embodiments, the population and the particles are initially contacted in a dry or non-hydrated form, e.g., after being subjected to a drying process, followed by adding a solution and contacting or culturing. In some embodiments, any one of the population or the particles is in a wet form and the other is in a dry form. In some embodiments, the particles are provided in dry form.

In some embodiments, a solution comprises an aqueous solution and a liquid solution.

The methods and compositions according to the invention, in some embodiments, have several advantageous properties (e.g., increased bacterial attachment to particles, increased bacterial load and/or increased capability to adhere to mucous surfaces), as demonstrated and detailed hereinbelow. In some embodiments, the particle-attached bacteria prepared according to the invention are resistant to environmental stress and persistence under unfavorable conditions in diverse environments. In some embodiments, the particle-attached bacteria prepared according to the invention are capable and/or configured to adhere to surfaces, e.g., for enabling a successful bacterial colonization in a subject administered therewith.

In some embodiments, the composition is characterized as having an increased number of bacteria attached to particles, e.g., when using or culturing in the presence of particles as detailed herein as compared to culturing in the presence of control particles (e.g., particles comprising microcrystalline cellulose in the absence of metal carbonate particles). In some embodiments, the increased bacterial attachment is in the range of 1.1 to 50-fold increase, e.g., at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 6.5-fold greater, at least 7-fold greater, at least 7.5-fold greater, at least 8-fold greater, at least 9-fold greater, at least 10-fold greater, at least 20-fold greater, at least 30-fold greater, at least 40-fold greater, at least 45-fold greater bacterial attachment as compared to control, or any value and range therebetween. Each possibility represents a separate embodiment of the invention.

In some embodiments, the terms “bacteria”, “a polysaccharide-based material”, “a metal carbonate” and “a metal phosphate” used throughout the description includes singular and plural references unless the context clearly specifies otherwise.

In some embodiment, the term “a population of at least one strain of bacteria” includes a population of at least one bacterial strain and a population of more than one bacterial strain.

In some embodiment, the terms “attached” and “adhered”, with regards to the interaction between the bacteria and the particles, are interchangeable and refer to adsorption of the bacteria to a surface/particle, e.g., via weak interactions and/or stronger interactions, e.g., by flagella, pili, lipopolysaccharides, exopolysaccharides, collagen-binding adhesive proteins, etc.

In some embodiments, the population of at least one strain of bacteria attaches to the particle during culturing. In some embodiments, the population of at least one strain of bacteria is grown in the presence of the particle.

In some embodiments, the invention provides a method of increasing the bacterial load, e.g., when using or culturing in the presence of particles as detailed herein as compared to using or culturing in the presence of particles comprising microcrystalline cellulose in the absence of metal carbonate particles and/or to planktonic culturing in the absence of particles. In some embodiments, the increased bacterial load at the end of culturing is in the range of 1.1 to 50-fold increase, e.g., at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 6.5-fold greater, at least 7-fold greater, at least 7.5-fold greater, at least 8-fold greater, at least 9-fold greater, at least 10-fold greater, at least 20-fold greater, at least 30-fold greater, at least 40-fold greater, at least 45-fold greater bacterial load as compared to using or culturing in the presence of particles comprising microcrystalline cellulose in the absence of metal carbonate particles and/or to planktonic culturing, or any value and range therebetween. Each possibility represents a separate embodiment of the invention.

In some embodiments, the composition according to the invention comprises CaCO₃ which advantageously possesses mucoadhesive properties, e.g., has an ability to be bonded and/or retained on mucous surface(s), such as through interaction with mucin. In some embodiments, the composition according to the invention is capable and/or configured to adhere to mucous surfaces (such as to skin, gut or vaginal epithelial cells), e.g., for enabling a successful bacterial colonization in a subject administered therewith. In some embodiments, the composition is configured to be bonded and/or retained on mucous surface(s) and release the bacteria from the composition at the target area for a prolonged period of time. For example, bacteria that is beneficial for skin, vaginal or gut treatment can be included in the composition, and the bacteria can be retained and released at the target area without being washed away for maximum therapeutic effect.

In some embodiments, the method of the present invention comprises a step of partial or complete elimination/decomposition/dissolvement of the metal carbonate particles. In some embodiments, the viability and beneficial effect, e.g., functionality of the bacteria is substantially maintained following the elimination or decomposition step. In some such embodiments, the composition comprises a decreased content of inert materials and an increased bacterial content, e.g., as compared to the ratio prior to the elimination step. Typically, the term “inert material” refers to a substance which has no biological activity for the intended use of the composition. In some embodiments, substantially maintaining the viability, beneficial effect and/or functionality of the bacteria refers to maintaining at least 80% of the initial value prior to the elimination/decomposition step. In some embodiments, between 80% and 100% of the initial value prior to the elimination/decomposition step is maintained.

In some embodiments, bacterial viability is determined by any method known to the skilled person such as, but not limited to, viable bacterial colony forming unit (CFU), live-dead staining, Propidium Monoazide qPCR (PMA-qPCR), a metabolic assay, spectrophotometry, or any combination thereof. Methods for determining bacterial viability, such as those disclosed herein, are common and would be apparent to a skilled artisan.

In some embodiments, the beneficial effect is determined by measuring the ability of bacteria to influence metabolic processes of a subject and/or ameliorate any disease, disorder or condition in a subject; measuring the potential metabolic routes/pathways of the bacteria, e.g., the potential to produce, synthesize, consume and/or utilize certain metabolites/organic compounds.

In some embodiments, the invention also provides a method of increasing bacterial resistance and a composition comprising resistant bacteria that can withstand harsh environmental and extreme manufacturing conditions such as antibiotic-resistant, acid-resistant, heat-resistant, and cold-resistant bacteria, e.g., as compared to bacteria grown in other growth forms.

In some embodiments, the composition disclosed herein is administered to a subject in need prior to, concomitant with, together with or following administration of an antibiotic and/or acidic substance. In some embodiments, the resilient/resistant bacterial population prepared according to the invention is co-administered or co-processed with antibiotic and/or acidic substance. In some embodiments, the antibiotic/acidic substance is released or administered prior to the beneficial/probiotic bacteria, thereby reducing the pathogen's abundance in the target tissue, and contributing to a successful colonization of the delivered beneficial or probiotic bacteria. Such combination treatments can be of advantageous, for example, in preventing or treating conditions of the urogenital tract of women that are characterized by increased pH levels and/or increase in the abundance of pathogenic bacteria. One non-limiting example of such a condition is Bacterial Vaginosis, wherein administration of a Lactic Acid producing bacteria prepared according to the invention, with or without antibiotic and/or an acidic substance, can be beneficial. Accordingly, in some embodiments, the composition according to the invention is used for treating Bacterial Vaginosis with or without antibiotic and/or an acidic substance. The antibiotic and/or the acidic substance can be co-administered with the composition according to the invention simultaneously, sequentially, or alternatingly. In some embodiments, the antibiotic is any antibiotic used for treatment of Bacterial Vaginosis. Non-limiting examples of antibiotics include metronidazole (Flagyl), clindamycin (Cleocin), and metronidazole. In some embodiments, an acidic substance comprises a pH adjusting agent. Non-limiting examples of pH adjusting agents according to the present invention are sodium bicarbonate, ascorbic acid, citric acid, acetic acid, fumaric acid, propionic acid, malic acid, succinic acid, gluconic acid, tartaric acid, lactic acid, boric acid cranberry extract and any combination thereof.

In some embodiments, extreme manufacturing conditions is, for example, a temperature of higher than 37° C., such as higher than 38° C., higher than 39° C., higher than 40° C., higher than 45° C., higher than 50° C., in the range of: higher than 37° C. to 40° C., higher than 37° C. to 45° C., higher than 37° C. to 50° C., 38° C. to 50° C., or in the range of 50° C. to 55° C.

In some embodiments, the composition comprises: a population of at least one strain of bacteria at least partially attached to particles, wherein the particles comprise: (i) a polysaccharide-based material; and (ii) a metal carbonate.

In some embodiments, the composition and/or method comprises a plurality of particle types or forms. In some embodiments, the composition and/or method, e.g., during contacting or culturing, comprise at least one of: (a) particles in a form of a composite comprising the polysaccharide-based material and the metal carbonate; and (b) particles comprising only one of: (i) the polysaccharide-based material or (ii) the metal carbonate. By “particles comprising only one of: (i) the polysaccharide-based material or (ii) the metal carbonate” it is meant that the composition or method comprises particles comprising polysaccharide-based material without metal carbonate and particles comprising metal carbonate without polysaccharide-based material. In some embodiments, “particles comprising only one of: (i) the polysaccharide-based material or (ii) the metal carbonate” means that the polysaccharide-based material and the metal carbonate are not combined, not processed together, not bonded, not linked and not connected to each other, e.g., via processing and/or any chemical interaction. In some embodiments, the terms “particles comprising only one of: (i) the polysaccharide-based material or (ii) the metal carbonate” and “comprising each of said polysaccharide-based material and said metal carbonate within a different particle” are interchangeable. In some embodiments, particles characterized as, a method or a composition “comprising each of said polysaccharide-based material and said metal carbonate within a different particle” means that the polysaccharide-based material and the metal carbonate are not included within the same particle, meaning that the polysaccharide-based material and the metal carbonate are not combined, not processed together, not bonded, not linked and not connected to each other, e.g., via processing and/or any chemical interaction. In such embodiments, a particle may comprise only one of: (i) the polysaccharide-based material or (ii) the metal carbonate and optionally additional material(s), e.g., the composition and/or method comprise particles comprising polysaccharide-based material without metal carbonate and particles comprising metal carbonate without polysaccharide-based material. In some embodiments, particles comprising polysaccharide-based material without metal carbonate; and particles comprising metal carbonate without polysaccharide-based material are suspended in the solution during contacting or culturing in close proximity to each other. In some embodiments, each particle type comprises at least one additional material. In some embodiments, the composition and/or method further comprise additional types of particles. In some embodiments, the composition and/or method further comprise other particle types devoid of both polysaccharide-based material and a metal carbonate, e.g., particles comprising dicalcium phosphate (DCP). The other particle types can be a blended or hybrid particle. In some embodiments, the composition and/or method comprise a plurality of different or identical composite particles, e.g., particles comprising polysaccharide-based material and metal carbonate and/or particles comprising dicalcium phosphate (DCP) and polysaccharide-based material. In some embodiments, a particle consisting essentially of metal carbonate is considered as a composite particle.

In some embodiments, the metal carbonate particle is not being produced by the at least one strain of bacteria. In some embodiments, the composition and/or method comprises metal carbonate being produced by at least one strain of bacteria. In some embodiments, the metal carbonate is formed or produced prior to culturing of the at least one strain of bacteria. In some embodiments, the metal carbonate is a water-insoluble metal carbonate. In some embodiments, the metal is provided in a non-consumable form and/or is water insoluble. In some embodiments, the metal carbonate is not being produced by a bacterial biofilm. In some embodiments, the metal carbonate is not being produced by the particle-attached bacteria of the invention. In some embodiments, the polysaccharide-based material is provided in a non-consumable form. In some embodiments, the polysaccharide-based material is not being produced by the at least one strain of bacteria, e.g., is not a self-produced exopolysaccharides. In some embodiments, the composition and/or method comprises polysaccharide-based material being produced by at least one strain of bacteria. In some embodiments, the polysaccharide-based material is formed or produced prior to culturing of the at least one strain of bacteria. In some embodiments, the polysaccharide-based material is water-insoluble. In some embodiments, the polysaccharide-based material is not being produced by the particle-attached bacteria of the invention. In some embodiments, at least one bacterial strain produces metal carbonate and/or polysaccharide-based material. In some embodiments, the particle is not being produced from a media precipitation.

In some embodiments, the method for preparing the composition comprises the steps of: providing a population of at least one strain of bacteria; providing particles, wherein the particles comprise: (i) a polysaccharide-based material; and (ii) a metal carbonate; contacting the population of at least one strain of bacteria with the particles, wherein the contacting is carried out in a solution; and allowing the population of at least one strain of bacteria to at least partially attach to the particles. In some embodiments, during the contacting or culturing step, the suspended particles have a characteristic selected from: (a) are in a form of a composite comprising the polysaccharide-based material and the metal carbonate; (b) comprise only one of: (i) the polysaccharide-based material or (ii) the metal carbonate; or both (a) and (b). In some embodiments, particles comprising only one of: (i) the polysaccharide-based material or (ii) the metal carbonate comprise: particles comprising the polysaccharide-based material without the metal carbonate and particles comprising the metal carbonate without the polysaccharide-based material.

As used herein, the term “a population of at least one strain of bacteria” refers to any integer equal to or greater than 1. In some embodiments, the composition comprises a population of one strain of bacteria and/or the method comprises contacting or culturing a population of one strain of bacteria. In some embodiments, a population of at least one strain comprises at least 2, at least 3, at least 4, at least 5, at least 6, at least 7, at least 8, at least 9, at least 10, at least 15, at least 20, at least 25, at least 30, at least 35, at least 40, at least 45, or at least 50 or more strains of bacteria. In the latter embodiment, the bacteria can be from one or more taxonomic classification.

In some embodiments, the population of at least one strain of bacteria originates or is derived from a biological sample. In some embodiments, the population of at least one strain of bacteria originates or is derived from human fluids, e.g., breast milk, vaginal fluids etc. In some embodiments, the population of at least one strain of bacteria is a synthetic sample. In some embodiments, the term “synthetic sample” refers to a sample comprising a bacterial community that is created artificially by combining/mixing selected (two or more) bacteria species.

As used herein, the terms “originated”, “derived”, and their conjugates are interchangeable with the term “obtained” and refer to the source from which the bacteria for culturing/fermenting are obtained.

In some embodiments, the population of at least one strain of bacteria originates or is derived from a fecal sample, an oral sample (e.g., a saliva sample), a skin sample, an eye sample, a bronchial sample, a vaginal sample, or any combination thereof. In some embodiments, the population of at least one strain of bacteria is derived from: a fecal bacterial population, a gut bacterial population, an eye bacterial population, an oral cavity bacterial population (e.g., a saliva bacterial population), a skin bacterial population, a bronchial bacterial population, a vaginal bacterial population, an upper respiratory tract bacterial population, a urogenital tract bacterial population, or any combination thereof. In some embodiments, the population of at least one strain of bacteria is derived from: soil bacterial population, ground water bacterial population, open waters bacterial population, or any combination thereof. In some embodiments, the population of at least one strain of bacteria is derived from plant(s). In some embodiments, the bacterial population originates or is derived from more than one bacterial source.

In some embodiments, the population comprises at least one mutant strain. As used herein, the term “mutant strain” embraces: addition, deletion, substitution, indel, inversion, duplication, or any combination thereof, e.g., compared to a naturally occurring bacterial strain (such as, but not limited to, a wildtype strain) or a genetic reference strain.

In some embodiments, the at least one strain of bacteria is a probiotic strain. In some embodiments, the term “probiotic” refers to a bacterial strain that when administered, e.g., in adequate amounts, can provide health benefits on the host (e.g., human) such as ameliorating any disease, disorder and/or condition in a subject, e.g., by improving or restoring healthy flora, influencing metabolic processes of a subject, and/or a stimulating the growth of other microorganisms, especially those microorganisms with beneficial properties.

In some embodiments, the population of at least one strain of bacteria comprises Gram+, Gram− bacteria, or both. In some embodiments, the population of at least one strain of bacteria comprises facultative anaerobe, tolerant anaerobe, obligate anaerobe, or any combination thereof. In some embodiments, the population of at least one strain of bacteria comprises obligate aerobe, microacrophiles, or any combination thereof. In some embodiments, the population of at least one strain of bacteria is isolated from a donor's sample. In some embodiments, a single isolated colony is selected from a donor's sample. In some embodiments, a complete sample is used in the method according to the invention, and the composition comprises a mixture of bacterial strains. In some embodiments, the population of at least one strain of bacteria comprises fecal microbiota, vaginal microbiota, e.g., originated from a collection of microorganisms colonizing a subject.

The bacterial strains may vary. In some embodiments, the population of at least one strain of bacteria belongs to a Lactobacilloae, Bifidobacteriaceae bacterial family. In some embodiments, the population of at least one strain of bacteria comprises a hydrogen peroxide (H₂O₂) producing bacteria species. In some embodiments, the population of at least one strain of bacteria comprises an ability to colonize an epithelial lined tissue.

In some embodiments, the population of at least one strain of bacteria comprises a Lactobacillus strain. In some embodiments, a Lactobacillus strain is characterized by or comprises an ability to colonize an epithelial lined tissue. In some embodiments, an epithelial lined tissue comprises a vaginal tissue. In some embodiments, the population of at least one strain of bacteria comprises at least one of the following species: Lactobacillus iners, Lactobacillus crispatus, Lactobacillus jensenii, Lactobacillus gasseri, Lactobacillus reuteri, Lactobacillus acidophilus, Lactobacillus vaginalis, Lactobacillus fermentum, Lactobacillus rhamnosus, Lactobacillus helveticus, Lactobacillus plantarum, Lactobacillus johnsonii, Lactobacillus paracasei, Lactobacillus salivarius, Lactobacillus delbrueckii, or any combination thereof. In some embodiments, the population of at least one strain of bacteria comprises at least one bacterium selected from: Lactobacillus crispatus, Lactobacillus gasseri, Lactobacillus jensenii, Lactobacillus rhamnosus, and any combination thereof. In some embodiments, the population of at least one strain of bacteria comprises Lactobacillus crispatus. In some embodiments, the population of at least one strain of bacteria comprises Lactobacillus rhamnosus. In some embodiments, the population of at least one strain of bacteria comprises Lactobacillus paracasei.

In some embodiments, the population of at least one strain of bacteria comprises at least one bacteria strain characterized by or comprises an ability to colonize a skin tissue. In some embodiments, the population of at least one strain of bacteria comprises at least one of the following species: Lactobacillus plantarum, Propionibacterium acnes, Corynebacterium tuberculostearicum, Propionibacterium acnes, Corynebacterium tuberculostearicum, Corynebacterium tuberculostearicum, Staphylococcus hominis, Staphylococcus epidermidis, Staphylococcus hominis, Streptococcus mitis, Propionibacterium acnes, Corynebacterium tuberculostearicum, Staphylococcus warneri, Streptococcus oralis, Staphylococcus epidermidis, Staphylococcus capitis, Staphylococcus epidermidis, Streptococcus pseudopneumoniae, Staphylococcus capitis, Corynebacterium simulans, Staphylococcus capitis, Streptococcus sanguinis, Corynebacterium fastidiosum, Streptococcus mitis, Staphylococcus haemolyticus, Micrococcus luteus, Corynebacterium afermentans, Staphylococcus hominis, Micrococcus luteus, Staphylococcus epidermidis, Micrococcus luteus, Corynebacterium aurimucosum, Corynebacterium afermentans, Staphylococcus capitis, Enhydrobacter aerosaccus, Corynebacterium kroppenstedtii, Corynebacterium simulans, Veillonella parvula, Corynebacterium simulans, Corynebacterium amycolatum, Corynebacterium resistens, Lactobacillus pentosus, Lactobacillus rhamnosus or any combination thereof. In some embodiments, the population of at least one strain of bacteria comprises Lactobacillus plantarum.

In some embodiments, the population of at least one strain of bacteria comprises a Staphylococcus strain, e.g., Staphylococcus epidermidis. In some embodiments, the population of at least one strain of bacteria comprises a Bifidobacterium strain, e.g., Bifidobacterium bifidum. In some embodiments, the population of at least one strain of bacteria comprises Escherichia coli Strain Nissle 1917.

In some embodiments, the phrase “bacteria at least partially attached to particles” refers to a range of between 0.01% and 100%, between 1% and 95%, between 5% and 90%, or between 10% and 85%, e.g., at least 0.01%, to at least 0.05%, at least 1%, at least 2%, at least 3%, at least 4%, at least 5%, at least 10%, at least 20%, at least 30%, at least 40%, at least 50%, at least 60%, at least 70%, at least 80%, at least 85%, at least 90%, at least 95%, at least 99%, or 100%, or any value and range therebetween, bacteria being in a particle-attached/adhered form out of the total bacteria present in the composition and/or at the end of the contacting or culturing step. In some embodiments, the bacteria are attached/adhered to at least one of: (i) a surface of the polysaccharide-based material; (ii) a surface of the metal carbonate material; or to both (i) and (ii). In some embodiments the majority of the attached bacteria are adhered to the surface of the metal carbonate particle (e.g., more than 50% and up to 100%, such as at least 51%, at least 55%, at least 60%, at least 65%, at least 70%, at least 75%, at least 80%, at least 85%, at least 90%, at least 95%, or even 100%, or any value and range therebetween out of the total attached bacterial fraction).

In some embodiments, “bacteria at least partially attached to particles” includes direct and indirect attachment or adherence of the bacteria to the particle. In some embodiments, an indirect attachment comprises attachment to the attached-bacteria and/or to a matrix formed by the attached/adhered bacteria.

In some embodiments, attachment can be determined by a scanning electron microscope (SEM) technique; by separating the attached and un-attached bacterial fractions, e.g., by filtration and/or gravitational sedimentation, e.g., centrifugation, and determining bacterial viability in the attached fraction; and/or by any other method known to the skilled in the art. The total bacteria present in the composition and/or at the end of the contacting or culturing step in both attached and un-attached fractions can be determined by any method known to the skilled person for bacterial viability such as disclosed herein. In some embodiments, the bacteria is separated from the particle prior to determining bacterial viability.

In some embodiments, bacterial attachment and percentage of bacterial attachment out of the total bacteria present within the culturing system is evaluated as follows: the particle-attached bacterial fraction is separated from the non-attached fraction by carrying out centrifugation (e.g., 3 min at 48×g; at 21±2° C.), and removing the supernatant comprising non-attached bacteria. In some embodiments, the pellet is washed with Saline 0.9%, centrifugation is repeated (e.g., 3 min at 48×g; at 21±2° C.), and the supernatant is removed, thereby obtaining a pellet of the particle-attached bacterial fraction. The pellet can be re-suspended with Saline 0.9% and drop assay can be carried out for the re-suspended fraction to evaluate bacterial viability in the attached fraction. Percentage of bacterial attachment can be evaluated by determining the bacterial viability in the total sample prior to its separation into the two fractions and calculating the bacterial viability in the attached fraction out of the viability in the entire sample.

In some embodiments, the term “particles” refers to a substance/material which is adapted, configured or suitable for attachment and/or growth thereto of at least one bacterial strain. In some embodiments, the particles comprise a surface to which the bacteria can adhere to. In some embodiments, in the context of the particles, the term “surface” is interchangeable with the term “surface area” and refers to the external surface of the particle. In some embodiments, the particles are porous. In some embodiments, the term “surface” comprises any outer surface of the porous structure of the particle including the inner voids of the particle. In some embodiments, the particles are non-porous. In some embodiments, the particles are a mix of porous and non-porous particles. In some embodiments, the term “porous” refers to the void (i.e., “empty”) spaces in a material. In some embodiments, the term “porous” includes having an un-even surface area. In some embodiments, the term “particles” refers to discrete three-dimensional shaped materials which are distinct, and separable from other substances/components added in the method, but this does not preclude components from being in contact with one another.

In some embodiments, when the population of at least one strain of bacteria originates or is derived from a biological sample, the particle does not originate from the sample. In some embodiments, when the population of at least one strain of bacteria originates or is derived from a biological sample, the sample is devoid of a particle as disclosed herein.

As used herein the term “particle(s)”, “nanoparticle(s)”, “microparticle(s)”, “nanosphere(s)”, and “microsphere(s)” are used interchangeably.

In some embodiments, all numerical percentages and w/w ratios herein relating to the particles, are based on the particles being in dry form. In some embodiments, the term “dry” with respect to the percentages and weight characteristics of the particles is interchangeable with the term “non-hydrated”. In some embodiments, the term “dry” refers to particles being subjected to a drying process. In some embodiments, the term “dry” refers to a water content within the particles that is lower than 5%. In some embodiments, the “dry particles” refers to particles that are free from un-bound water. In some embodiments, the particles comprise bound water. In some embodiments, wherein the particles are prepared by precipitation into the culture medium, the dry weight can be calculated by isolating the particles from the culture medium, e.g., by centrifugation, removing the liquid phase, and subjecting the isolated particles to a drying step until a water content of less than 5% is obtained. In some embodiments, the isolated particles are first subjected to a step of separating attached bacteria, e.g., by vortex. Water content can be determined according to the Karl Fischer titration, Loss on drying (LOD), and/or by any other method known to the skilled person.

In some embodiments, the particles are water-insoluble particles. In some embodiments, the particles comprise water-insoluble particles. In some embodiments, the term “water-insoluble” refers to a particle that substantially does not dissolve in water, e.g., saline, phosphate buffered saline, and a growth medium. In some embodiments, the term “substantially does not dissolve” means at least 90 wt. % of the particles, preferably 95 wt. % of the particles, does not dissolve in water. In some embodiments, the solubility of the particles in water is equal to or less than 2.5 g/1,000 ml at a temperature in the range of 20° C. to 60° C., e.g., at a temperature selected from 20, 25, 37, 60° C. and/or at a pH in a range of 5.0 to 8.0. Each possibility represents a separate embodiment of the invention. In some embodiments, the particle used in accordance to the invention is water-insoluble in a physiological pH. In some embodiments, the particle used in accordance to the invention is water-insoluble in pH ranging from about 1.0 to 8.0.

In some embodiments, the composition and/or method comprises a plurality of types of particles, e.g., having a different size distribution. In some embodiments, the particles range from 0.1 micron to 1 cm in diameter. In some embodiments, the particles range from 5 microns to 1 cm in diameter. In some embodiments, the particles range from 0.1 to 500 microns in diameter. In some embodiments, the particles range from 1 micron to 50 millimeters. In some embodiments, the composition and/or method comprise particles smaller than 1 micron in diameter. In some embodiments, the composition and/or method comprise particles in the range of 30 to 500 microns (e.g., in the range of 40 to 400 microns, 50 to 300 microns, 60 to 200 microns, 70 to 100 microns, or any value and range therebetween). In some embodiments, the composition and/or method comprise particles in the range of 0.2 to 100 micron (e.g., in the range of 1 to 90 microns, 1 to 80 microns, 1 to 70 microns, 1 to 60 microns, 1 to 50 microns, 1 to 40 microns, 1 to 30 microns, 1 to 25 microns, 1 to 20 microns, 2 to 90 microns, 4 to 80 microns, 6 to 70 microns, 8 to 60 microns, 10 to 50 microns, 12 to 40 microns, 14 to 30 microns, 16 to 20 microns, or any value and range therebetween), or any value and range therebetween. In some embodiments, the particles are at least 0.2 microns in diameter, at least 2 microns in diameter, at least 3 microns in diameter, at least 5 microns in diameter, at least 10 microns in diameter, at least 15 microns in diameter, at least 20 microns in diameter, at least 30 microns in diameter, at least 40 microns in diameter, at least 50 microns in diameter, at least 60 microns in diameter, at least 70 microns in diameter, at least 80 microns in diameter, at least 90 microns in diameter, at least 100 microns in diameter, at least 200 microns in diameter, at least at least 300 microns in diameter, at least 400 microns in diameter, at least 500 microns in diameter, at least 600 microns in diameter, at least 700 microns in diameter, at least 800 microns in diameter, at least 900 microns in diameter, at least 1 cm in diameter, or any value and range therebetween. Each possibility represents a separate embodiment of the invention.

In some embodiments, the average diameter of the particles is in the range of 0.2 to 1,500 microns. In some embodiments, the average diameter of the particles is in the range of 50 to 1,200 microns, 50 to 1,100 microns, 50 to 1,000 microns, 55 to 1,200 microns, 55 to 1,000 microns, 57 to 1,200 microns, or 60 to 1000 microns, including any range therebetween. Each possibility represents a separate embodiment of the invention.

In some embodiments, a diameter is an average diameter. In some embodiments, a diameter is a maximal diameter. In some embodiments, a diameter is a minimal diameter.

In some embodiments, the term “particles” includes any particle, e.g., in the embodied size range, in any shape. In some embodiments, the particle can be round, amorphous, irregular, sphere, elliptical, flower, cubic, spherical, elongated, rod-shaped, having any other shape, or any combinations thereof.

In some embodiments, at least a portion of the particles comprise a composite or a mixture comprising: (i) a polysaccharide-based material; and (ii) a metal carbonate. In some embodiments, during the contacting and/or culturing step the particles are in a form of a composite comprising the polysaccharide-based material; and the metal carbonate. In some embodiments, the composite particles are formed during the contacting and/or culturing step, e.g., via a chemical interaction. In some embodiments, the particles are provided into the method as a composite or a mixture comprising: (i) a polysaccharide-based material; and (ii) a metal carbonate.

In some embodiments, the term “at least a portion of the particles comprise a composite or a mixture comprising: (i) polysaccharide-based material; and (ii) metal carbonate” refers to a range of between 1% and 100%, between 5% and 90%, between 10% and 80%, or between 20% and 70%, e.g., at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or even 100% (on a weight basis), or any value and range therebetween, of the particles being in a form of a composite particle comprising both a polysaccharide-based material; and a metal carbonate. All percentages are out of the total particles present within the composition and/or provided, added or used in the method. In some embodiments, the remaining particles are in a form comprising only one of: polysaccharide-based material or a metal carbonate. In some embodiments, the “composite particles” and/or the “remaining particles” comprise additional type(s) of material(s).

As used herein the term “composite” or “mixture” refers to a particle that is composed of different materials. In some embodiments, a composite particle comprises: (i) a polysaccharide-based material; and (ii) a metal carbonate, and optionally additional material(s) or substance(s).

In some embodiments, a portion of the particles comprise only one of: the polysaccharide-based material or the metal carbonate. In some embodiments, the term “a portion” with relation to the particles comprising only one of: the polysaccharide-based material or the metal carbonate refers to the remaining percentage of particles in the composition and/or provided, added or used in the method.

In some embodiments, during the contacting and/or culturing step the particles are separated particles comprising only one of: the polysaccharide-based material or the metal carbonate. In some embodiments, the polysaccharide-based material; and the metal carbonate are provided, added or used in the method as distinct particles, each comprising only one of the polysaccharide-based material or the metal carbonate. In some embodiments, the term “distinct” comprises the terms “individual”, “separated”, “different” or “non-blended”, and relates to the particles' constitute with respect to the polysaccharide-based material, and the metal carbonate. In some embodiments, the “distinct particles” are blended or mixed with an additional material. In some embodiments, the distinct particles form a composite during and/or following contact with a solution.

In some embodiments, the particles in the composition and/or provided, added or used in the method comprising only one of: the polysaccharide-based material or the metal carbonate are less than 100%; such as less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 4%, less than 3%, less than 2%, less than 1% (on a weight basis), or any value and range therebetween. All percentages are out of the total particles within the composition and/or the particles provided, added or used in method.

In some embodiments, the majority (e.g., more than 50%, such as between 51% and 100%, at least 51%, at least 55%, at least 60%, at least 65%, at least 70%, at least 75%, at least 80%, at least 85%, at least 90%, at least 95%, or even 100% (on a weight basis), or any value and range therebetween) of the particles in the composition and/or the particles provided, added or used in the method are composite particles comprising a combination of at least a polysaccharide-based material; and a metal carbonate. All percentages are out of the total particles within the composition and/or method.

In some embodiments, the composition and/or method comprise a plurality of particle types, e.g., comprising different materials. In some embodiments, the composition and/or method comprise different types of polysaccharide-based materials, different types of metal carbonates, or any combination thereof. In some embodiments, each particle may comprise different types of polysaccharide-based materials and/or different types of metal carbonates.

In some embodiments, each particle comprises additional material(s), thereby forming “a blended or hybrid particle”. In some embodiments, the additional material comprises Dicalcium Phosphate (DCP). In some embodiments, each particle comprises one material. In some embodiments, each particle comprises two or more different materials. In some embodiments, the composition and/or method further comprises particles devoid of polysaccharide-based material and metal carbonate. In some embodiments, the composition and/or method comprises particles comprising DCP, a polysaccharide-based material, or a combination thereof.

In some embodiments, the materials are homogeneously dispersed within the individual particle. The term “homogenous” means that the materials are substantially uniformly dispersed throughout the individual particle, e.g., without being present in a substantially higher concentration in any part of the particle. In some embodiments, different regions of the particle have approximately the same material constitution. In some embodiments, different regions of the particle have different material constitution.

In some embodiments, the composite particles, comprising the polysaccharide-based material and the metal carbonate, have at least one characteristic selected from: (i) were not subjected to physical processing; (ii) were subjected to physical processing; (iii) are not a co-processed mixture; (iv) are a co-processed mixture; and (v) the polysaccharide-based material and the metal carbonate are bonded, linked or connected via a chemical interaction.

In some embodiments, the composition and/or method comprises blended or hybrid particles, e.g., comprising a mixture of various materials, having at least one characteristic selected from: (i) were not subjected to physical processing; (ii) were subjected to physical processing; (iii) are not a co-processed mixture; (iv) are a co-processed mixture; and (v) the materials composing the particle are bonded, linked or connected via a chemical interaction.

In some embodiments, the composition and/or method comprises a plurality of composite, blended or hybrid particles comprising any combination of characteristics (i) to (v).

In some embodiments, physical processing comprises compression, shear force or the like. In some embodiments, particles in a form of a co-processed mixture comprises materials/substances that are combined by applying mechanical pressure.

In some embodiments, the composite, blended or hybrid particles are added in the method as separated materials, and form the composite, blended or hybrid particle, e.g., via any chemical bond, force and/or interaction.

In some embodiments, the method further comprises a step of dispersing the polysaccharide-based material and the metal carbonate in a solution. In some embodiments, the polysaccharide-based material and the metal carbonate are dispersed together in the same solution or in separated solutions, followed by the step of combining the two dispersions. In some embodiments, the dispersing step is carried out in the same solution, simultaneously, sequentially, and/or alternatingly. In some embodiments, the method further comprises a step of mixing the solution(s) comprising the dispersed polysaccharide-based material and metal carbonate, e.g., by a simple blend. In some embodiments, a simple blend comprises conventional mixing or blending operations such as shaking, stirring and the like. In some embodiments, the composite particles comprising the polysaccharide-based material and the metal carbonate are formed by dispersing the two separated particle types in a solution and/or by mixing/combing two separated dispersed solutions each comprising a different particle type/material. Non-limiting examples of solutions are saline solution, phosphate buffer solution, a growth medium and the like. In some embodiments, dispersing is carried out prior to, simultaneously with, and/or following inoculating or adding the bacteria into the solution. In some embodiments, following blending, the solution is a homogeneous mixture of the dispersed particle types. In some embodiments, one or more additional particle type(s) is dispersed in the solution. In some embodiments, the polysaccharide-based material and the metal carbonate, and optionally the additional particle type, are dispersed in the solution simultaneously, one after the other, or both. In some embodiments, the particles/materials are dispersed in the solution in dried form, e.g., powdered form, in wet form, or both.

In some embodiments, at least a portion of the particles are blended, hybrid or composite particles comprising a mixture of more than one substance. In some embodiments, a portion of the particles comprise a mixture of a polysaccharide-based material; and a metal carbonate. In some embodiments, the composition and/or method comprise particles comprising a polysaccharide-based material. In some embodiments, the composition and/or method comprise particles comprising a metal carbonate. In some embodiments, the composition and/or method comprise particles comprising only one of: a polysaccharide-based material or a metal carbonate, and optionally an additional material. In some embodiments, the composition and/or method comprise particles comprising a polysaccharide-based material and substantially no metal carbonate. In some embodiments, the composition and/or method further comprise particles comprising a metal carbonate and substantially no polysaccharide-based material. In some embodiments, the composition and/or method comprise particles consisting essentially of: (i) a polysaccharide-based material and a metal carbonate; (ii) a polysaccharide-based material; (iii) metal carbonate; or (iv) any combination of (i) to (iii).

In some embodiments, the weight ratio between: (i) particles comprising a polysaccharide-based material and substantially no metal carbonate: (ii) particles comprising a metal carbonate and substantially no polysaccharide-based material: and (iii) composite particles comprising a polysaccharide-based material, and a metal carbonate is in a range of between 1:1:1 and 10:10:10, e.g., a ratio of 1:1:10, 10:1:1, 1:10:1, 1:10:10, 10:10:1, 10:1:10, or any value and range therebetween. All ratios are based on weight per weight per weight (w/w/w) basis of the dry particles form.

In some embodiments, “a polysaccharide-based material” refers to materials comprising a polysaccharide, whereas the term “polysaccharide” denotes a pure carbohydrate, such as starch or cellulose. In some embodiments, “a polysaccharide-based material” refers to naturally occurring polymers composed of glucose units, e.g., connected by a 1-4 beta glycosidic bond. In some embodiments, the polysaccharide-based material is a linear polysaccharide, e.g., materials that form crystalline or partially crystalline structures, e.g., at a temperature in the range of 20 to 60° C., e.g., at a temperature selected from 20, 25, 37, 60° C. and/or at a pH in a range of 5 to 8. In some embodiments, the term “a polysaccharide-based material” comprises oligosaccharide-based materials.

In some embodiments, the polysaccharide-based material comprises materials/substances based on cellulose; pectin; inulin; dextrin; maltodextrin; glycogen; starches, e.g., derived from corn or potato; amylose; alginate; chitosans, e.g., hydrophobically modified chitosans (HMCs); fructooligosaccharide (FOS); galactooligosaccharide (GOS); or any combination thereof.

In some embodiments, the polysaccharide-based material comprises fructooligosaccharide (FOS); galactooligosaccharide (GOS) or a combination thereof. In some embodiments, cellulose-based substances comprise materials comprising cellulose fibers such as microcrystalline cellulose (MCC).

In some embodiments, the polysaccharide-based material is characterized by having a positive net electrical charge, a negative net electrical charge or neutral net electrical charge. In some embodiments, the polysaccharide-based material comprises surfaces having an electrical charge. As used herein the term “net electrical charge” refers to the overall electric charge of the material. For example, a positively net electrical charged material is a material that has an overall excess of protons. In an embodiment, wherein the composition and/or method comprise a plurality of types of polysaccharide-based materials, each type of polysaccharide-based material may have a different net electrical charge. In some embodiments, the plurality of types of polysaccharide-based materials are characterized by having a positive net electrical charge, a negative net electrical charge, a neutral net electrical charge or any combination thereof.

In some embodiments, the polysaccharide-based material is negatively charged, e.g., MCC is negatively charged, at a pH ranging between 3.5 and 10.0.

In s embodiments, “a metal carbonate” is interchangeable with the term “mineral salt” and refers to an inorganic compound comprising a metal cation and a carbonate anion (CO₃⁻²). Typically, a metal carbonate can undergo decomposition, e.g., upon reaction with an acid, to produce salt, water, and carbon dioxide gas (CO₂).

In some embodiments, the metal ions/positively charged metal ions/metal cations are in the form monovalent, divalent, and trivalent metal ions. In some embodiments, the composition and/or method comprise a plurality of types of metal carbonates comprising a plurality of types of metal cations. In some embodiments, the metal cations comprise monovalent, divalent, trivalent metal cations or any combination thereof.

In some embodiments, the metal carbonate is or comprises divalent cation(s). In some embodiments, the metal cation is or comprises Calcium (Ca²⁺), Barium (Ba²⁺), Manganese (Mn²⁺), Iron (Fe²⁺), Magnesium (Mg²⁺), Zinc (Zn²⁺), or any combination thereof. In some embodiments, the metal cation comprises Calcium (Ca²⁺).

In some embodiments, the metal cation is or comprises calcium cation (Ca²⁺), and the metal carbonate is or comprises calcium carbonate (CaCO₃). In some embodiments, the polysaccharide-based material is or comprises cellulose derivative(s), e.g., microcrystalline cellulose (MCC). In some embodiments, at least a portion of the particles in the composition and/or method are in a form of a composite particle comprising a mixture of: (i) MCC; and (ii) calcium carbonate. In some embodiments, a portion of the particles in the composition and/or method comprise (i) MCC; and (ii) calcium carbonate as separated, non-blended particles. In some embodiments, each particle further comprises an additional material.

“Calcium carbonate” (CaCO₃) that can be used according to the invention can be synthesized or manufactured in any method known in the art, e.g., for obtaining nanometer and micrometer/micron materials, and is typically available in several forms, shapes and crystalline structure. In some embodiments, the form and type used according to the invention is suitable for human consumption. In some embodiments, the calcium carbonate is derived from natural source, synthetic source, or a combination thereof.

In some embodiments, the metal carbonate comprising particles, e.g., calcium carbonate, are precipitated particles. In some embodiments, the metal carbonate used in accordance with the invention are pre-made particles, e.g., are commercially available. In some embodiments, the precipitated particles are formed within the vessel used for contacting the bacteria with the particles. In some embodiments, the method further comprises a step of precipitating, e.g., dropping, calcium oxide into a solution, e.g., contained within a culturing vessel; thereby providing the particles comprising the calcium carbonate. In some embodiments, the method further comprises a step of precipitating, e.g., dropping dry Na₂CO₃ and CaCl₂) into a solution and/or mixing solutions of Na₂CO₃ and CaCl₂), thereby providing the particles comprising the calcium carbonate. In some embodiments, a solution is water, saline, phosphate buffered saline, or a growth medium. In some embodiments, the calcium carbonate particles are purified, refined and/or synthetic calcium carbonate. In some embodiments, the calcium carbonate comprising particles are ground calcium carbonate. In some embodiments, the calcium carbonate comprises impurities.

In some embodiments, the average diameter of the calcium carbonate particles is in the range of 0.2 to 100 microns, e.g., 2-20 microns, 2.1 to 3 microns, 0.2 to 5 microns, 20 to 100 microns, or any value and range therebetween. In some embodiments, the calcium carbonate comprises amorphous CaCO₃, crystalline CaCO₃, or any combination thereof. In some embodiments, the calcium carbonate comprises a crystalline phase selected from the group consisting of: calcite (β-CaCO₃), aragonite (λ-CaCO₃), vaterite (μ-CaCO₃); and any combination thereof.

In some embodiments, the metal carbonate and the polysaccharide-based material are present during contacting and/or culturing in a concentration that will allow the at least partial attachment of the population to the particles.

Without being bound by a particular theory or mechanism, using high metal carbonate concentrations yields a viscous culturing medium that may require an increase in the stirring speed to obtain a homogenous culturing media, thereby negatively affecting bacterial attachment to the particles; and using low metal carbonate concentrations, will also result in a lower bacterial attachment.

Accordingly, in some embodiments, the total weight of the particles with respect to a volume of the solution, e.g., a growth medium, during the contacting and/or culturing step is up to 250 g per 1 L. In some embodiments, the total weight of the particles with respect to a volume of the solution, e.g., a growth medium, during the contacting and/or culturing step is at least 60 g per 1 L. In some embodiments, the total weight of the particles with respect to a volume of the solution, e.g., a growth medium, during the contacting and/or culturing step is in a range of between 60 g per 1 L and 250 g per 1 L, e.g., between 60 g per 1 L and 200 g per 1 L.

In some embodiments, in such particles: solution ratio, the metal carbonate is at most 85% w/w out of the total weight of the particles. In some embodiments, in such particles: solution ratio, the metal carbonate constitutes up to 85% w/w out of the total weight of the particles. In some embodiments, in such particles: solution ratio, the metal carbonate constitutes up to 75% w/w out of the total weight of the particles. In some embodiments, in such particles: solution ratio, the metal carbonate is at most 75% w/w out of the total weight of the particles. In some embodiments, in such particles: solution ratio, the metal carbonate constitutes at least 15% w/w out of the total weight of the particles. In some embodiments, in such particles: solution ratio, the metal carbonate constitutes at least 25% w/w out of the total weight of the particles. In some embodiments, in such particles: solution ratio, the metal carbonate content ranges between 15% and 85% w/w out of the total weight of the particles. In some embodiments, in such particles: solution ratio, the metal carbonate content ranges between 25% and 75% w/w out of the total weight of the particles. All percentages are w/w out of the total weight of the particles provided, added or used in the method. Accordingly, in some embodiments, the metal carbonate constitutes at least 15% w/w out of the total weight of the particles within the composition. In some embodiments, the metal carbonate constitutes at least 25% w/w out of the total weight of the particles within the composition. In some embodiments, the metal carbonate constitutes up to 85% w/w out of the total weight of the particles within the composition. In some embodiments, the metal carbonate is at most 85% w/w out of the total weight of the particles within the composition. In some embodiments, the metal carbonate constitutes up to 75% w/w out of the total weight of the particles within the composition. In some embodiments, the metal carbonate is at most 75% w/w out of the total weight of the particles within the composition. In some embodiments, the metal carbonate content ranges between 25% and 75% w/w out of the total weight of the particles within the composition. In some embodiments, the metal carbonate content ranges between 15% and 85% w/w out of the total weight of the particles within the composition.

In some embodiments, the metal carbonate content in the composition and/or in the particles provided, added or used in the method ranges between 15% to 85% (w/w), 15% to 80% (w/w), 15% to 75% (w/w), 15% to 70% (w/w), 15% to 65% (w/w), 15% to 60% (w/w), 15% to 55% (w/w), 15% to 50% (w/w), 15% to 45% (w/w), 15% to 40% (w/w), 20% to 85% (w/w), 25% to 75% (w/w), 25% to 85% (w/w), 25% to 80% (w/w), 20% to 50% (w/w), 20% to 48% (w/w), 20% to 45% (w/w), or 20% to 40% (w/w) out of the total weight of the particles in the composition and/or the particles provided, added or used in the method.

In some embodiments, the remaining percentages are particles comprising polysaccharide-based material. In some embodiments, the polysaccharide-based material content ranges between 15% to 85% (w/w), 15% to 80% (w/w), 15% to 75% (w/w), 15% to 70% (w/w), 15% to 65% (w/w), 15% to 60% (w/w), 15% to 55% (w/w), 15% to 50% (w/w), 15% to 45% (w/w), 15% to 40% (w/w), 20% to 85% (w/w), 25% to 75% (w/w), 25% to 85% (w/w), 25% to 80% (w/w), 20% to 50% (w/w), 20% to 48% (w/w), 20% to 45% (w/w), or 20% to 40% (w/w) out of the total weight of the particles in the composition and/or the particles provided, added or used in the method.

In some embodiments, the weight of the provided metal carbonate particles with respect to the volume of the solution is at least 9 g per 1 L. In some embodiments, the weight of the provided metal carbonate particles with respect to the volume of the solution is up to 215 g per 1 L. In some embodiments, the weight of the provided metal carbonate particles with respect to the volume of the solution is in a range of between 9 g per 1 L and 215 g per 1 L, 9 g per 1 L and 200 g per 1 L, 15 g per 1 L and 190 g per 1 L.

In some embodiments, the weight of the provided polysaccharide-based material particles with respect to the volume of the solution is at least 9 g per 1 L. In some embodiments, the weight of the provided polysaccharide-based material particles with respect to the volume of the solution is up to 215 g per 1 L. In some embodiments, the weight of the provided polysaccharide-based material particles with respect to the volume of the solution is in a range of between 9 g per 1 L and 215 g per 1 L, 9 g per 1 L and 200 g per 1 L, 15 g per 1 L and 190 g per 1 L.

In some embodiments, the polysaccharide-based material, e.g., MCC; and the metal carbonate, e.g., calcium carbonate, are present in the composition and/or in the solution during the contacting or culturing step in a weight per weight (w/w) ratio ranging from 1:10: to 10:1, 1:6 to 6:1, or any value and range therebetween. In some embodiments, the polysaccharide-based material; and the metal carbonate are present in a weight per weight (w/w) ratio of 6:1, 5:1, 4:1, 3:1, 2:1, 1:1, 1:2, 1:3, 1:4, 1:5, 1:6, or any value and range therebetween. Each possibility represents a separate embodiment of the invention.

In some embodiments, the particles further comprise metal phosphate. In some embodiments, the composite particle further comprises metal phosphate. In some embodiments, the metal phosphate comprises dicalcium phosphate (DCP). In some embodiments, “a metal phosphate” refers to an inorganic compound comprising a metal cation and a phosphoric acid.

In some embodiments, when the particles further comprise metal phosphate, the metal phosphate, and the metal carbonate constitute together at least 15% (w/w) out of the total weight of the particles. In some embodiments, the ratio between metal phosphate and the metal carbonate ranges between 1:14 to 14:1, e.g., 2:13, 4:11, 6:9, 8:7, 13:2, 11:4, 9:6, 7:8, 2:13, or any value and range therebetween. Each possibility represents a separate embodiment of the invention. In some embodiments, when the particles further comprise metal phosphate, the metal phosphate, and the metal carbonate constitute together at least 25% (w/w) out of the total weight of the particles. In some embodiments, the ratio between metal phosphate and the metal carbonate ranges between 1:24 to 24:1, e.g., 7:18, 9:16, 11:14, 13:12, 18:7, 16:9, 14:11, 12:13, 7:18, or any value and range therebetween. Each possibility represents a separate embodiment of the invention.

In some embodiments, when the total weight of the particles with respect to a volume of the solution, e.g., a growth medium, during the contacting and/or culturing step is lower than 60 g per 1 L, the metal carbonate concentration can be higher than 85% w/w out of the total weight of the particles. In such an embodiment, the metal carbonate concentration can be up to 99% w/w out of the total weight of the particles. In other embodiments, the metal carbonate concentration is at least 15% w/w out of the total weight of the particles. In other embodiments, the metal carbonate concentration is at least 25% w/w out of the total weight of the particles. In some embodiments, the metal carbonate concentration is between 15% and 99% w/w out of the total weight of the particles, e.g., between 15% and 95%, between 15% and 90%, between 20% and 95%, between 25% and 99%, between 25% and 90%, or any value and range therebetween. Each possibility represents a separate embodiment of the invention. All percentages are w/w out of the total weight of the particles provided, added or used in the method.

Accordingly, in some embodiments, the metal carbonate constitutes at least 15% w/w out of the total weight of the particles within the composition. In some embodiments, the metal carbonate constitutes at least 25% w/w out of the total weight of the particles within the composition. In some embodiments, the metal carbonate constitutes up to 99% w/w out of the total weight of the particles within the composition. In some embodiments, the metal carbonate content ranges between 15% and 99% w/w out of the total weight of the particles within the composition, e.g., between 15% and 95%, between 15% and 90%, between 20% and 95%, between 25% and 99%, between 25% and 90%, or any value and range therebetween. Each possibility represents a separate embodiment of the invention.

In some embodiments, the CaCO₃ is at least partially entrapped and/or adsorbed by the polysaccharide-based material.

In some embodiments, the metal carbonate, e.g., CaCO₃, and the polysaccharide-based material are linked, adsorbed, fused, or superficially connected to each other, e.g., by any chemical bond, force and/or interaction.

In some embodiments, the method comprises contacting a population of at least one strain of bacteria with particles; and allowing the population of at least one strain of bacteria to at least partially attach to the particles.

The phrase “contacting” is used herein in its broadest sense and refers to any type of combining action which, e.g., brings the bacteria into proximity with the particles such that the bacteria can attach/adhere to the particles. In some embodiments, contacting comprises incubating, growing or culturing the bacteria with or in the presence of the particles, e.g., as opposed to planktonic culturing carried out in the absence of particles. In some embodiments, contacting comprises combining the bacteria, the particles, and the solution in any order, any combination and/or sub-combination including any pre-mixing of two of the elements prior to adding the third element, or simultaneously mixing all elements. In some embodiments, the method comprises a step of inoculating the bacteria in a solution, and thereafter culturing the bacteria in a solution comprising the particles. In some embodiments, the method comprises a step of providing a solution comprising the particles as a preceding step and then adding the population of at least one strain of bacteria. In some embodiments, contacting and culturing are carried out simultaneously or as subsequent steps.

In some embodiments, contacting or culturing is carried out in a solution. In some embodiments the solution is selected from the group consisting of: a buffer, saline, phosphate buffered saline, and a growth medium. In some embodiments, contacting comprises culturing the bacteria in the presence of the particles.

In some embodiments, the term “culturing” includes the term “fermentation” and refers to the in-vitro maintenance, proliferation and/or growth of cells, e.g., bacterial cells, on or in buffers and/or media of various kinds under laboratory or industrial conditions. In some embodiments, the proliferated or grown cell may not be completely, morphologically, genetically, or phenotypically identical to the parent cell. The term “fermentation” has its ordinary meaning in the art. In some embodiments, the term “fermentation” is used herein to refer to a microbiological metabolic process comprising conversion of sugar(s) to acids and/or gases using, e.g., bacteria.

A suitable medium is apparent to the person skilled in the art and may be any known or commercially available medium, or otherwise may be synthesized and/or custom designed, e.g., to support the maintenance, proliferation and/or growth of the cultured cells. Non-limiting examples of media include, but are not limited to, Tryptic Soy Broth (TSB), Yeast Casitone Fatty Acids (YCFA), Robertson's Cooked Meat, Reinforced Clostridium medium (RCM) broth, nutrient broth, Fastidious Anaerobic Broth (FAB), Lysogeny broth (LB), ¼ LB, Heart infusion broth, Wilkins-Chalgren broth, Brucella broth, wheat bran, Brain Heart Infusion (BHI), Brain heart infusion agar (BHI1), M9 minimal media or 0.2×BHI, Gifu Anacrobic Medium (GAM), Gut Microbiota Medium (GMM), Columbia blood agar (CBA), Chocolate agar (CHOC), Tryptic soy agar (TSY), Fastidious anacrobe agar (FAA), Tryptic Yeast Extract Glucose (TYG), Cooked meat agar (BEEF), Bifidobacterium Selective Media (BSM), Phenylethyl alcohol agar (PEA), Actinomyces isolation agar (AIA), Colistin naladixic acid agar (CNA), Mckay agar (MK), Mannitol slat agar (MSA), de Man Rogosa Sharpe agar (MRS), Bacteroides bile esculin agar (BBE), Deoxycholate agar (DOC), Rogosa Agar, CDC ANAEROBIC BLOOD AGAR (CDC), MacConkey agar (MAC), Staphylococcus Medium, Bifido Medium and Kanamycin vancomycin laked blood agar (KVLB), or any combination and equivalents thereof. In some embodiments, the media comprises pharma-grade ingredients. In some embodiments, the media comprises food-grade ingredients. In some embodiments, the media comprises components suitable for veterinary use.

In some embodiments, the method comprises the steps of providing the bacteria contained within a vessel, and contacting or culturing is carried out within the vessel. In some embodiments, the term “vessel” refers to any receptacle wherein bacteria can be cultured in conventional fermentation techniques, e.g., bioreactor(s), flask(s), test tube(s), microtiter dish(es), well-plate(s), multi-well plate assembly, petri-plate(s), and the like.

In some embodiments, the method further comprises a step of inoculating the population of at least one strain of bacteria in a solution, e.g., as a preceding step to the contacting step. In some embodiments, the contacting comprises a step of inoculating the population of at least one strain of bacteria in a solution. In some embodiments, contacting comprises a step of incubating the population of at least one strain of bacteria in the solution comprising the particles for a time sufficient for the at least one strain of bacteria to at least partially attach to the particles. In some embodiments, the contacting step comprises culturing the population of at least one strain of bacteria, e.g., in a growth medium. In some embodiments, contacting comprises inoculating a solution comprising particles with the bacteria; and incubating or culturing the bacteria with the particles for a time sufficient to allow the bacteria to at least partially attach to the particle.

In some embodiments, contacting or culturing is carried out under conditions that allow the population of at least one strain of bacteria to at least partially attach to the particles. In some embodiments, a suitable condition comprises pH level, e.g., in a range of 5 to 8, under flow, under shake, under stir, under agitation, under static, moist, low humidity, or any combination thereof. In some embodiments, a suitable condition comprises selecting a medium that supports the growth of the cultured bacterial strain(s). In some embodiments, a suitable condition is contacting or culturing the population of at least one strain of bacteria with the particles for a period in a range of from 1 hour to 15 days. In some embodiments, a time sufficient to allow the bacteria to at least partially attach/adhere to the particle ranges from: 2 hours to 10 days, 2 hours to 8 days, 2 hours to 6 days, 2 hours to 4 days, 2 hours to 2 days, 2 hours to 24 hours, 6 hours to 24 hours, 10 hours to 24 hours, 15 hours to 20 hours, 20 hours to 40 hours, 6 hours to 40 hours, or any value and range therebetween. Each possibility represents a separate embodiment of the invention.

In some embodiments, contacting or culturing is carried out under conditions comprising: static, flow, stirring, shaking, agitating or any combination thereof, e.g., at 50 to 750 revolutions per minute (RPM), 50 to 650 RPM, 100 to 750 RPM, 100 to 700 RPM, 150 to 700 RPM, 200 to 750 RPM, 130 to 690 RPM, 90 to 720 RPM, 70 to 550 RPM, 110 to 710 RPM, or any value and range therebetween. Each possibility represents a separate embodiment of the invention. In some embodiments, contacting is carried out under static conditions. In some embodiments, contacting is carried out under static conditions, followed by contacting under flow conditions. In some embodiments, contacting is carried out under flow conditions, followed by contacting under static conditions.

In some embodiments, the bacteria is contacted with the particles or cultured under shear force conditions. In some embodiments, the solution, e.g., growth medium, exerts a shear force on the bacteria during the contacting or culturing step. In some embodiments, the term “shear force” refers to the movement of the solution in relation to the bacteria, e.g., to the bacteria being attached to a particle. In some embodiments, shear force conditions comprises contacting or culturing the bacteria under conditions comprising: mixing, rocking, flow, rolling, vortexing, stirring, shaking, agitating, foaming, pumping, bubble formation, or any combination thereof.

In some embodiments, the terms “flow”, “stirring”, “shaking” and “agitating” refer to conditions leading to movement or motion of the liquid phase within the culturing vessel. In some embodiments, the movement is axial, radial, mixed, distributed, or any combination thereof. The movement or motion can be carried out by using a mechanical mean, e.g., an impeller, a moving platform, rocker, shaker; manually; automatically or any combination thereof. In some embodiments, the term “static conditions” refers to conditions where no agitation or any other motion action (either manually, automatically and/or mechanically) is performed on the liquid phase.

In some embodiments, the contacting or the culturing step comprises culturing under anaerobic conditions. In some embodiments, the term “anaerobic conditions” refer to conditions wherein free oxygen is lower than 500 ppm, 450 ppm, 400 ppm, 350 ppm, 300 ppm, 250 ppm, 200 ppm, 150 ppm, 100 ppm, 50 ppm, or 10 ppm, or any value and range therebetween. Each possibility represents a separate embodiment of the invention. In some embodiments, anaerobic conditions comprise conditions providing no free oxygen. In some embodiments, anaerobic conditions comprise conditions being devoid of free oxygen.

In some embodiments, contacting or the culturing step comprises culturing under aerobic conditions. In some embodiments, the term “aerobic conditions” refers to conditions comprising the presence of molecular oxygen. In some embodiments, the oxygen concentration is above 20% (v/v out of the total gas present during culturing).

In some embodiments, the growth medium further comprises elements that enhances bacterial attachment to the particles.

In some embodiments, after attachment of the bacteria to the particle(s), some or all of the bacteria can be detached, and/or other bacteria in suspension can attach to the particle, particle-attached/adhered bacteria and/or to a matrix formed by the attached/adhered bacteria.

In some embodiments, the method comprises a step of separating the attached and non-attached bacterial fractions, e.g., to produce a composition comprising attached bacteria; and a composition comprising non-attached bacteria. In some embodiments, the method further comprises a step of removing from the solution, e.g., culturing or growth medium, bacteria unattached to the particles. In some embodiments, the step of separating or removing is carried out at at-least one time point selected from the group consisting of: prior to, during, after the incubating, contacting or culturing step, and any combination thereof. In some embodiments, unattached/unadhered bacteria are or comprise planktonic bacteria. In some embodiments, unattached bacteria are devoid of sessile bacteria.

In some embodiments, separating the fractions or removal of unattached bacteria is carried out by or comprises filtration and/or gravitational sedimentation, e.g., centrifugation. In some embodiments, the term “filtration” includes all separation techniques as well as any other processes utilizing a filter that can separate the fractions.

In some embodiments, the composition comprises particle-attached bacteria and/or bacteria in planktonic form. In some embodiments, the composition comprises bacteria attached to a particle. In some embodiments, the composition comprises bacteria in planktonic form.

In some embodiments, the method comprises a step of eliminating the metal carbonate from the particles, the particle-attached bacterial fraction and/or from the solution wherein contacting or culturing is carried out; thereby creating a composition comprising trace amounts of the metal carbonate.

In some embodiments, the step of eliminating the metal carbonate is carried out at at-least one time point during the preparation method. In some embodiments, the elimination step is carried at a time point selected from: (i) during contacting, e.g., during culturing; (ii) at the end of the contacting step, e.g., at the end of culturing; (iii) prior to separation of the particle-attached bacteria from the non-attached bacterial fraction; (iv) following separation of the particle-attached bacteria from the non-attached bacterial fraction; or (v) at any time point combination of (i) to (iv).

In some embodiments, the elimination step is carried out within the vessel wherein contacting is carried out and/or in a different vessel. In some embodiments, elimination is carried out in the particle-attached bacteria. In some embodiments, the elimination step is carried out in a precipitated fraction comprising the particle-attached bacteria. In some such embodiments, the precipitate can be re-suspended prior to the elimination, e.g., with saline.

In some embodiments, the term “eliminating the metal carbonate” includes partial or complete decomposition of the metal carbonate, e.g., depending on the amount of acid and/or CO₂ added to the solution during the contacting step, e.g., during the culturing step. In some embodiments, between 1% and 100% (on a weight or mole basis) of the initial calcium carbonate particles are eliminated. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or even 100%, or any value and range therebetween, of the initial calcium carbonate particles are eliminated.

In some embodiments wherein the particles comprise metal phosphate, e.g., DCP, adding acid and/or CO₂ may result in partial or complete elimination of the DCP from the particles, the bacterial attached fraction and/or from the solution wherein contacting or culturing is carried out.

In some embodiments, elimination comprises adding an acid to the solution. In such an embodiment, carbon dioxide gas (CO₂), metal salt and water are produced. For example, in an embodiments wherein: (i) calcium carbonate (CaCO3) is the metal carbonate compound, and (ii) the acid is HCl→carbon dioxide, calcium chloride (CaCl₂)) and water are produced. In some embodiments, the carbon dioxide gas is released through the solution and out of the vessel wherein contacting is carried out. In another embodiment wherein: (i) magnesium carbonate (MgCO3) is the metal carbonate compound, and (ii) the acid is HCl→carbon dioxide, magnesium chloride (MgCl₂) and water are produced. In some embodiments, the carbon dioxide gas is released through the solution and out of the vessel wherein contacting is carried out.

In some embodiments, the term “metal salt” refers to a compound comprised of at least one anion and at least one cation. In some embodiments, the formed salt is solid at room temperature, and is water-soluble at the temperature wherein contacting is carried out.

In some embodiments, following elimination of the metal carbonate, the free metal ion is removed from the solution by adsorption, absorption, chelation, or any combination thereof.

In some embodiments, the term “acid” as used herein refers to an acid in the meaning of the definition by Brønsted and Lowry. In some embodiments, the acid comprises acetic acid. In some embodiments, the acid is a halogen acid such as hydrogen fluoride (HF), hydrogen chloride (HCl), hydrogen bromide (HBr), hydrogen iodide (HI), hydrogen astatide (HAt), hydrogen tennesside (HTs); or any combination thereof. In some embodiments, the acid comprises HCl, HBr and HI.

In some embodiments, elimination comprises creating a CO₂ enriched environment in the solution wherein contacting is carried out. In such an embodiment, wherein calcium carbonate (CaCO3) is the metal carbonate compound, calcium carbonate reacts with the carbon dioxide and water to produce calcium hydrogen carbonate [(Ca(HCO₃)₂].

In some embodiments, elimination comprises contacting the composition with an effective amount of a chelator or a chelating agent.

In some embodiments, following the elimination/decomposition step, the composition, the particles and/or the solution wherein the contacting is carried out comprises trace amounts of metal carbonate. In some embodiments, during and/or following the elimination step, an increase in the level of any of the decomposition products can be detected by any method known to the skilled person, e.g., measuring salt concentration, carbon dioxide and/or calcium hydrogen carbonate levels in the vessel or solution wherein the contacting is carried out.

In some embodiments, the composition comprises trace amounts of the metal carbonate. In some embodiments, the term “trace amount” refers to a concentration on a range of 0.01 percent to lower than about 5 percent. In some embodiments, the term “trace amount” refers to a concentration of lower than about 5 percent, e.g., lower than 4 percent, lower than 3 percent, lower than 2 percent, lower than 1 percent, or any value and range therebetween, of metal carbonate out of the total weight of the composition. In some embodiments, the term “trace amount” refers to a concentration of lower than about 1 percent of metal carbonate out of the total weight of the composition.

In some embodiments, partial or complete dissolution of the metal carbonate particles, e.g., by acid and/or CO₂, can be obtained on a mole calculation of the acid and/or CO₂ added into the solution relative to the moles of the metal ion present within the particles as can be recognized by the skilled in the art. In some embodiments, complete dissolution of the metal carbonate particles can be obtained by adding acid into the solution in a mole ratio that equals the moles of the metal ion present within the particles. In an embodiment wherein: (i) calcium carbonate (CaCO3) is the metal carbonate compound, (ii) the acid is HCl, and iii) complete dissolution is desired, the mole to mole ratio between HCl and calcium carbonate can be 2:1. In some embodiments, the mole to mole ratio between HCl and calcium carbonate is lower and can be, e.g., 1.5:1, or 1.25:1 or lower. In some embodiments, the acid is added in an amount that does not affect the viability of the bacteria, e.g., the pH level during the dissolution is suited to enable proliferation, growth and/or maintenance of the bacteria.

In some embodiments, the composition further comprises bacteria in aggregated form, e.g., following decomposition of the metal carbonate particles. In some such embodiments, the majority of the attached bacteria are adhered to the surface of the polysaccharide-based material (e.g., more than 50%, such as at least 51%, at least 55%, at least 60%, at least 65%, at least 70%, at least 75%, at least 80%, at least 85%, at least 90%, at least 95%, or even 100%, or any value and range therebetween out of the total attached bacterial fraction). The total attached bacteria can be determined by separating the two fractions as disclosed hereinabove.

In some embodiments, the term “bacteria in aggregated form” is interchangeable with the term “bacterial clumps” and refers to the collection of individual bacterial particles into a single body. In some embodiments, the composition according to the invention comprises an increased content of aggregated bacteria as compared to a composition prepared under the same contacting/culturing conditions and in the absence of a particle and/or in the presence of currently employed particles for bacterial growth, e.g., at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 6.5-fold greater, at least 7-fold greater, at least 7.5-fold greater, at least 8-fold, at least 9-fold, at least 10-fold, or higher or any value and range therebetween.

In some embodiments, the composition comprises bacteria in the form of biofilm. In some embodiments, the biofilm is in the form of dried biofilm or in a solid form, e.g., in powder form. In some embodiments, the term “biofilm” refers to a community of bacteria embedded within a matrix comprising self-produced exopolysaccharides, e.g., that adheres to a particle's surface. In some embodiments, planktonic bacteria are stuck, entrapped, merged, or embedded, under, onto, or within the biofilm.

In some embodiments, the bacterial load of the composition; or at the end of the contacting or culturing step is at least 1·E⁴ such as at least 1·E⁶ per 1 g, e.g., per 1 g particle provided, added or used in the method according to the invention; per the total weight of the bacteria at the end of culturing and/or per the total weight of the composition. In some embodiments, the method further comprises a step of harvesting the cultured bacteria. In some embodiments, the harvested cultured bacteria have a bacterial load of at least 1·E⁴ such as at least 1·E⁶ per 1 g, e.g., per 1 g particle and/or per the total weight of the harvested bacteria.

In some embodiments, the bacterial load ranges between at least 1·E⁴ to at least 1·E²⁰, at least 1·E⁵ to at least 1·E¹⁵, at least 1·E⁵ to at least 1·E¹⁰, or at least 1·E⁷ to at least 1·E⁹. In some embodiments, the bacterial load is at least 1·E⁶, at least 1·E⁷, at least 1·E⁸, at least 1·E⁹, at least 1·E¹⁰, at least 1·E¹¹, at least 1·E¹², at least 1·E¹⁴, at least 1·E¹⁶, or at least 1·E²⁰, or any value and range therebetween. Each possibility represents a separate embodiment of the invention. In some such embodiments, the bacterial load can be per 1 g, e.g., per 1 g particle provided, added or used in the method, per the total weight of the cultured bacteria at the end of, and/or 1 g composition.

In some embodiments, determining bacterial load is carried out according to any standard method available and known to a person or ordinary skill in the art, non-limiting examples of which include, but are not limited to, plate count methods, spectrophotometric (turbidimetric) analyses, or others.

In some embodiments, bacterial load is determined by viable bacterial colony forming unit (CFU), quantitative polymerase chain reaction (qPCR), flow-cytometry, live-dead staining, Propidium Monoazide qPCR (PMA-qPCR), microscopy, a metabolic assay, other common methods, or any combination thereof.

In some embodiments, the method further comprises contacting or culturing at least one additional microorganism. In some embodiment, the composition further comprises at least one additional microorganism. In some embodiments, the at least one additional microorganism is selected from: fungi, bacteriophages, viruses, archaea, and the like, or any combination thereof, e.g., which originate or are derived from a biological sample, and are found, constitute or known to reside in an environmental niche of a subject.

In some embodiments, the particles are dispersed within the composition or within a carrier comprising the composition. In some embodiments, with relation to the presence of the particles within the composition or carrier the term “dispersed” comprises particles being present substantially throughout the composition or carrier without being present in a substantially higher concentration in any part of the composition or carrier. Additionally, the term “dispersed” also comprises particles being present in localized areas of the composition or carrier.

In some embodiments, the composition and/or method according to the invention comprises a population of a single strain of bacteria or mixtures of two or more strains. In such some embodiments, the different strains are contacted or cultured with the particles separately, together as a co-culture; or a mixture of both. The bacterial strain and/or ratio between different strains may vary, e.g., depending on the intended use. In some embodiments, strains and ratios may be selected according to desirable characteristics of the strain(s), e.g., the ability to reduce the PH level at the target area, inhibit growth of specific pathogenic bacteria etc. In some embodiments, the different strains are isolated/originated from different sources, e.g., different donors. In some embodiments, when the bacteria originate from a sample, e.g., donor(s), the composition and/or culturing method may be enriched or supplemented with additional bacteria.

According to some embodiments, there is provided a composition produced according to the method of the invention.

According to some embodiments, there is provided a pharmaceutical composition comprising the composition disclosed herein and an acceptable carrier or excipient.

In some embodiments, the term “pharmaceutical composition” includes the term “a dietetic composition”, and “nutritional composition”.

In some embodiments, the terms “a dietetic composition”, “nutritional composition”, and “nutraceutical composition” refer to a composition that is suited for being consumed as a food supplement, e.g., for supplementing the normal diet, correcting nutritional deficiencies, maintaining an adequate intake of certain nutrients, and/or for supporting specific physiological functions.

In some embodiments, the composition according to the invention is administered to a subject immediately after culturing. In some embodiments, the composition is administered following storage, e.g., at room temperature, at a temperature in the range of 2-8° C. or a temperature below −18° C. In some embodiments, the composition is provided in a solid form, e.g., lyophilized, spray-dried or frozen state. In some embodiments, the solid compositions disclosed herein are stable at room temperature (e.g., at a temperature selected from the group consisting of: about 20, 21, 22, 23, 24, and 25° C.) for a period of at least three months. In this context, the term “solid” refers to the physical state of a material. In some embodiments, the composition is a solid composition. In some embodiments, the composition is a lyophilized composition.

Typically, the term “lyophilization” and “freeze-dried” are interchangeable and refer to the process of freezing a solution followed by reducing the concentration of water, e.g., by sublimation to levels that do not support biological and/or chemical reactions. The resulting lyophilized composition can be stored for a long period of time while maintaining its stability. In some embodiments, the lyophilized composition can be used as a solid. In some embodiments, the solid or composition may be disposed in a suitable delivery vehicle such as fat-based carriers, e.g., for use as suppository, or can be reconstituted by the addition semi-liquid or a liquid solution. The volume added during the reconstitution can be similar, lower, or higher as compared to the initial volume of the solution before the lyophilization process.

In some embodiments, the composition is for pharmaceutical use. In some embodiments, the composition is for agricultural use. In some embodiments, the composition is for veterinary use. In some embodiments, the composition is for use as a non-prescription medicine, e.g., as a supplement.

In some embodiments, the pharmaceutical composition is for use in the treatment or prevention of dysbiosis in a subject in need thereof. In some embodiments, the composition as disclosed herein is for use in the manufacture of a medicament for the treatment or prevention of dysbiosis related condition.

In some embodiments, the composition is a synthetic composition. In some embodiments, the term “synthetic composition” refers to a composition comprising in-vitro grown or cultured bacteria. In some embodiments, a synthetic composition comprises an artificial composition. In some embodiments, a synthetic composition is a man-made composition, such as, but not limited to, a composition made or produced in a laboratory and/or a manufacturing site or facility. In some embodiments, a synthetic composition does not include a composition isolated or obtained from nature per se. In some embodiments, the composition is frozen, spray-dried or freeze-dried. In some embodiments, the composition is in the form of a dried powder. In some embodiments, the composition comprises: cryoprotectant, lyoprotectant, an anti-oxidant, or any combination thereof. In some embodiments, the composition comprises at least one metabolite that is produced in-vitro by at least one bacterium.

In some embodiments, there is provided a method for preventing or treating dysbiosis in a subject in need, comprising administering to the subject a therapeutically effective amount of a composition according to the invention.

The term “a therapeutically effective amount” or “an effective amount” refers to the amount required to prevent, ameliorate and/or treat a disease, disorder, or condition. The effective dose may be changed depending on the gender, age and weight of the subject, the disease or condition and its severity and on any other factor which can be recognized by the skilled in the art.

The compositions and formulations disclosed herein can be used internally and externally, e.g., for the treatment or prevention of dysbiosis. In some embodiments, the compositions and formulations can be administered to a surface of a body part of a subject in need thereof.

As used herein the term “a surface of a body part of a subject” refers to an external surface of the body that can be seen by unaided vision, e.g., the skin of the face, throat, scalp, chest, back, car, neck, hand, elbow, knee, and other skin sites; and to a surface of an internal body part, e.g., which is a part of the internal anatomy of an individual, such as, but not limited to, the oral cavity, the gastrointestinal tract and the lower genital tract including, but not limited to the vagina.

As used herein, the term “dysbiosis” is characterized by altered, imbalance, impairment and/or dysfunction of the microbiota in any tissue and/or surface of any body part of the organism, including, but not limited to, any external or internal surface of the body and deep layers of the skin which is typically associated with a disease, health condition or a clinical symptom. The imbalance can be in any microbial community, including, without any limitations, a gastrointestinal tract, skin, oral, bronchial, vaginal, or rectal imbalance to name a few.

In some embodiments, the dysbiosis is a tumor dysbiosis. In some embodiments, the composition is used for treating gastrointestinal or gut dysbiosis. In some embodiments, the composition is used for treating skin and tissue dysbiosis. In some embodiments, the composition is used for treating dysbiosis related to interstitial cystitis, polycystic ovary syndrome. In some embodiments, the dysbiosis is vaginal and female genital dysbiosis, such as associated with bacterial vaginosis, urinary tract infections, a human papilloma virus infection, urogenital infection, urinary tract infection, a candidiasis infection, infertility, a sexually transmitted infection, and a gynecological cancer. In some embodiments, the composition is used for treating male genital dysbiosis. In some embodiments, the composition is used for reducing and/or preventing dysbiosis related to preterm birth, and miscarriages.

In some embodiments, dysbiosis comprises imbalance of microbial flora. In some embodiments, the pharmaceutical composition is for use in modulating a flora in a subject in need thereof. In some embodiments, the composition as disclosed herein is for use in the manufacture of a medicament for modulating a flora. In some embodiments, there is provided a method for modulating a flora in a subject in need, comprising administering to the subject a therapeutically effective amount of a composition according to the invention. In some embodiments, microbial flora comprises a vaginal flora, a gut flora, or a skin flora.

In some embodiments, the term “modulating a flora” comprises suppressing or reducing the abundance of bacteria which is typically associated with a disease, health condition or a clinical symptom; restoring a healthy microbiome balance; increasing diversity and/or achieving colonization of a beneficial bacteria in any tissue and/or surface of any body part of an organism, including, but not limited to, any external or internal surface of the body and deep layers of the skin which are typically associated with a disease, health condition or a clinical symptom.

In some embodiments, the term “treating dysbiosis” comprises generating a desired/predetermined microbial profile in an environmental niche of a subject that is beneficial, e.g., for increasing responsiveness of the subject to an administered drug.

In some embodiments, “microbial profile” comprises a bacterial diversity (e.g., α-diversity or β-diversity); bacterial relative abundance; and/or bacterial load.

In some embodiments, the terms “bacterial load”, “bacterial viability”, and “bacterial count” are interchangeable. In some embodiments, the composition comprises viable bacteria and the bacterial count is measured by CFU.

In some embodiments, the bacterial load in the composition produced according to the invention is calculated per gr dry-form particle introduced into the culturing medium or vessel. In some embodiments, the bacterial load is calculated per gr dry-form particle used to contact with the bacteria. In some embodiments, when measuring the bacterial load of the composition, the weight of the particles added to the culturing system is considered. In some embodiments, when measuring the bacterial load of the bacteria in the composition, the weight of the particles in the final composition is considered.

In some embodiments, the altered microbiota comprises pathogenic microbes. In some embodiments, an altered microbiota is associated with a medical condition and/or is harmful to a subject's health, e.g., a human subject.

As used herein, the term “altered microbiota”, refers to a flora that has diverted from the homoeostatic microbiota, such as in a non-healthy subject.

In some embodiments, the subject is a mammal. In some embodiments, the subject is an animal. In some embodiments, the subject is a human subject. The subject may be male or female.

In some embodiments, a subject in need of treatment, as described herein, is afflicted with or at risk of being afflicted with a disease, disorder, or condition.

In some embodiments, the composition comprises a carrier or an excipient. In some embodiments, the carrier is a veterinary, an agricultural and/or pharmaceutical acceptable carrier. In some embodiments, the terms “carrier”, “excipient” or “adjuvant” refer to any component of a pharmaceutical composition that is not the active agent. As used herein, the term “pharmaceutically acceptable” carriers, solvents, diluents, excipients, and vehicles generally refer to non-toxic, inert solid, semi-solid, liquid filler, diluent, encapsulating material, formulation auxiliary of any type, or simply a sterile aqueous solution, such as saline. In some embodiments, the term a “pharmaceutically acceptable carrier” refers to any diluent or a vehicle which is suitable for human or other animal use. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes and hard fats; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g., carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369. The carrier may comprise, in total, from about 0.1% to about 99.99999% by weight of the pharmaceutical compositions presented herein.

A pharmaceutical composition may take any physical form necessary for proper administration. The composition can be administered in any suitable form, including but not limited to, a liquid form, a gel form, a semi-liquid (e.g., a liquid, such as a viscous liquid, comprising some solid) form, a semi-solid (a solid comprising some liquid) form, or a solid form. Compositions can be provided in, for example, a tablet form, a pessary form, a cream form, a suppository form, a capsule form, a liquid form, a food form, a chewable form, a non-chewable form, a transbuccal form, a sublingual form, a slow-release form, a non-slow-release form, a sustained release form, or a non-sustained-release form.

In some embodiments, the composition is formulated for administration by a mode selected from the group consisting of: rectal, parenteral, mucosal, vaginal, nasal, local, topical, pulmonary, ocular, oral, buccal administration, and any combination thereof. In some embodiments, the composition is administered orally. In some embodiments, the composition is administered locally (e.g., by inserting a suppository, capsule or any other appropriate form into the vagina).

In some embodiments, the composition may be disposed in various drug-delivery systems, including but not limited to, a capsule, suppository, soluble shell, and the like.

In some embodiments, the composition is provided in a form of liquid or semi-solid, cream, suppository, pessary, tablet, pill, caplet, capsule, granules, ointment, lotion, gel, spray, sachet, liquid drops, oral liquids, suspension, syrup, emulation, oil, or film.

In some embodiments, the composition comprises: a carrier; and at least on cultured bacterial species disposed within the carrier. In some embodiments, the composition comprises a mixture of dry bacteria. In some embodiments, each bacterium is cultured separately. In some embodiments, the composition comprises co-cultured bacteria.

In some embodiments, the composition is disposed within an enteric coating.

In some embodiments, the composition is configured for pH dependent targeted release of the bacterial strain in the gastrointestinal tract. In some embodiments, the composition is covered with a compound, e.g., alginate, and is configured to release the bacteria at a pH found in the intestine of a subject.

### General

All numeric values are herein assumed to be modified by the term “about”. The term “about” generally refers to a range of numbers that a person skilled in the art would consider equivalent to the recited value (e.g., having the same function or result). In many instances, when a numerical value is preceded by the term “about”, the term “about” is intended to indicate ±10%.

The terms “comprises”, “comprising”, “includes”, “including”, “having” and their conjugates mean “including but not limited to”.

The term “consisting of” means “including and limited to”.

The term “consisting essentially of” means that the composition, method, or structure may include additional ingredients, steps and/or parts, but only if the additional ingredients, steps and/or parts do not materially alter the basic and novel characteristics of the claimed composition, method, or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an”, and “the”, include plural references unless the context clearly dictates otherwise. As used in this specification and the appended claims, the term “or” is generally employed in its sense including “and/or” unless the content clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. For example, a description of “a range between 15% and 85%” should be considered as including the first and second indicated percentages and all numbers within the indicated range. The phrases “at least”, “up to”, and “at most” used herein are meant to include the indicated number. For example, “up to 85%” should be considered as including the indicated number.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, microbiological and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., cd. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transciption and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference. Other general references are provided throughout this document.

In the Examples below, culturing of various bacterial strains having different characteristics, e.g., different taxonomic groups and different origins, as elaborated under Table 1 below, was carried out as an embodiment, to exemplify the superiority of the method according to the invention to produce a composition comprising a population of at least one strain of bacteria at least partially attached to particles, when using particles as indicated herein vs. other culturing methods. In the Examples, MCC and CaCO₃ were used as embodiments for a polysaccharide-based material and metal carbonate particles.

### Materials and Methods

### Tested Bacteria

TABLE 1
Tested bacteria.

| | | Species & | |
|---|---|---|---|
| Family | Genus | Source | Additional Characteristics |
| Lacto- | Lacto- | Lacto- | Gram+; facultative anaerobic; |
| bacillaceae | bacillus | bacillus | rod-shaped species; located in |
| | | crispatus | both the vagina, and in the |
| | | (DSM | vertebrate gastrointestinal |
| | | 20584) | tract. |
| | | | Is commonly used for the |
| | | | prevention and treatment of |
| | | | Bacterial Vaginosis, which is |
| | | | characterized by the low |
| | | | abundance of Lactobacillus |
| | | | flora necessary to protect the |
| | | | host from infection. |
| | | Lacto- | Gram+; oxygen-tolerant |
| | | bacillus | anaerobe; rod-shaped (bacillus |
| | | paracasei | shape) bacterium; typically |
| | | (DSM | located in the intestinal tract |
| | | 5622) | and mouth. |
| Bifido- | Bifido- | Bifido- | Gram+; obligate anaerobe; |
| bacteriaceae | bacterium | bacterium | neither motile nor spore- |
| | | bifidum | forming; is rod-shaped and |
| | | (DSM | can be found living in |
| | | 2045) | clusters, pairs, or even |
| | | | independently. Most of the |
| | | | population of Bifidobacterium |
| | | | bifidum is found in the colon, |
| | | | lower small intestine, breast |
| | | | milk, and often in the vagina. |
| Lacto- | Lactiplanti- | Lacto- | Gram+; facultative anaerobic; |
| bacillaceae | bacillus | bacillus | rod-shaped species; a nomadic |
| | | plantarum | organism that can be found in |
| | | (Isolated | various environments |
| | | from adult | including vertebrate intestine, |
| | | human feces) | the skin. |

### Particles Used

Microcrystalline cellulose (MCC); and CaCO₃ (CAS #: 1-34-471) were used. All particles' weight and ratio values within the Examples section refer to the particles being in dry, non-hydrated form. The water content within the particles was lower than 5%. The particles can be in a range from 0.1 to 500 microns in diameter.

### Culturing Conditions and Preparation of a Bacterial Composition

Culturing was carried out as detailed below under “Experimental procedure” in a sterile bottle containing 200 ml of growth medium with or without 40 g particles of: (i) MCC; or (ii) MCC and CaCO₃ at different ratios (w/w).

MCC and CaCO₃ tested ratios were: 75%: 25% (30 g: 10 g); 50%: 50% (20 g: 20 g); and 25%: 75% (10 g: 30 g).

100% CaCO₃ or 100% MCC particles were also tested.

Alternatively, a particle mixture comprising MCC/CaCO₃/DCP can be used.

The medium used for culturing included: For Lactobacillus crispatus and Lactobacillus paracasei—Yeast extract; Peptone; Tween; L-cysteine; Sodium, Potassium, Manganese and Magnesium salts; and + (D)-Glucose. For Bifidobacterium bifidum—Bifidobacterium broth supplemented with L-cysteine; Tween; and + (D)-Glucose.

Planktonic growth (culturing in the absence of particles) was also carried out under corresponding conditions as elaborated for each bacterium.

In case culturing was carried out in the presence of particles, the particles were pre-mixed with the medium prior to mixing with the inoculum (the pH of the medium was adjusted according to the bacterium used—see below conditions).

### Experimental Procedure

A culture of each bacterium was prepared from an initial inoculum stored at −80° C. (the stock inoculum contained planktonic bacteria with glucose 50% solution in a ratio of 5:1 w/v).

Fifty (50) μl (for Lactobacillus crispatus and Lactobacillus paracasei) or 100 μl (for Bifidobacterium bifidum) inoculum was mixed with 200 ml medium with or without 40 g particles (100% MCC; 100% CaCO₃ or MCC/CaCO₃ mixtures as specified above), and incubated/cultured for 10 hours (for Lactobacillus crispatus and Lactobacillus paracasei) or 20 hours (for Bifidobacterium bifidum) under the following conditions: pH in the range ˜5.0-6.5; and a Temperature of 37° C. in anaerobic conditions (Gas mix: N₂—90%, H₂—5%, CO₂—5%) with stirring at about 25 RPM using a magnet for the entire culturing period.

In the Examples, CaCO₃ particles were eliminated from the bacterial composition by adding an acid solution.

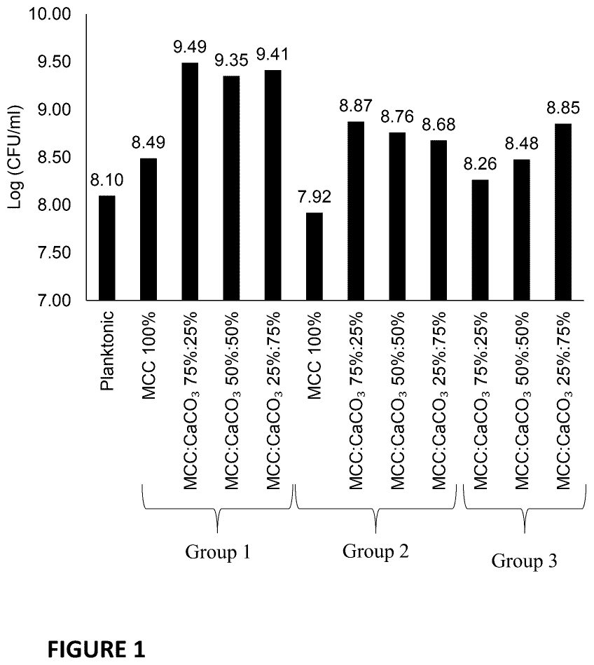

For quantitatively assessing the bacterial attachment to the different particles tested, evaluation of the total bacterial count [presented in Log (CFU/ml)] was carried out as follows:
at the end of the culturing for: (i) planktonic growth, i.e., culturing without particles (FIG. 1, “Planktonic”); and (ii) for culturing in the presence of particles (for the total bacterial mass, i.e., combined particle-attached and non-attached bacterial fractions; appears in FIG. 1 as “Group 1”). Planktonic culturing was used as a reference group; and
in the separated particle-attached bacterial fraction (appears in FIG. 1 as “Group 2”). The separation procedure for each tested group was carried out in the manner elaborated below.
   Comparing the bacterial count in the different attached fractions (appearing in Group 2), provides an indication for any changes in the level of attachment when using a particle mix according to the invention vs. culturing in the presence of MCC without CaCO₃ particles.

For assessing the feasibility of particle dissolution using an acid, evaluation of the bacterial count (CFU) was carried out after elimination/titration of the CaCO₃ particles from the particle-attached bacterial fraction (appears in FIG. 1 as “Group 3”).

The results in FIG. 1 show the bacterial count (Log CFU/ml) obtained in Groups 1-3 and in planktonic culturing for Bifidobacterium bifidum.

All methodologies were carried out as specified below.

### Bacterial Load Measurements (Colony Forming Unit, CFU)

Unless indicated otherwise, CFU measurements were carried out as follows: A sample was taken from each culturing bottle and transferred into three sterile tubes (3 tubes for each culturing condition; 10 ml for each).

For Group 1, for removing bacteria bound to particles, vortex was carried out for 2 minutes at high speed. To assess the attachment, CFU measurement (drop assay) was carried out from each tested condition by serial dilutions, and bacteria were plated in triplicate onto agar plates. The plates were incubated at 37° C. in anaerobic conditions (Gas mix: N₂—90%, H₂—5%, CO₂—5%) for 24-48 h prior to CFU counting.

For Group 2, the particle-attached bacterial fraction was separated from the non-attached fraction by carrying out centrifugation (3 min at 48×g; at 21±2° C.), followed by removal of the supernatant, and washing the pellet containing the particle-attached bacterial fraction with Saline 0.9%. Centrifugation was repeated under the same conditions, and the supernatant was removed, thereby obtaining a pellet (about 5 g) of the particle-attached bacterial fraction. The pellet was re-suspended with 5 ml of Saline 0.9% and drop assay was carried out as elaborated above for Group 1.

For Group 3, the particle-attached bacterial fraction was separated from the non-attached fraction by carrying out centrifugation (3 min at 48×g; at 21±2° C.), followed by removal of the supernatant, and washing the pellet containing the particle-attached bacterial fraction with Saline 0.9%. Centrifugation was repeated under the same conditions, and the supernatant was removed, thereby obtaining a pellet (about 5 g) of the particle-attached bacterial fraction. The pellet was re-suspended with 5 ml of Saline 0.9%, CaCO₃ particles were eliminated with HCl as elaborated below, and drop assay was carried out as elaborated for Group 1.

For planktonic growth, aggregates were detached/separated by vortex for 2 minutes at high speed, and drop assay was carried out.

### CaCO₃ Particle Dissolution/Elimination Using an Acid

For Lactobacillus crispatus, Lactobacillus paracasei and Bifidobacterium bifidum, 5 M HCl was gradually added to the Saline (0.9%) re-suspended fraction of Group 3, prepared as elaborated above, in portions of 100-200 μl, while manually mixing the sample. The exact volume added to each culturing condition is indicated in Table 2.

Generally, HCl was added taking into consideration two criteria:
1—CO₂ release from the solution, indicating an ongoing CaCO₃ particle dissolution reaction; and
2—pH level throughout the dissolution step, to assure viability and functionality of the bacteria at the end of the CaCO₃ particle dissolution.

Several HCl volumes were tested and the volumes listed in Table 2 represent maximal CaCO₃ particle dissolution while minimally affecting bacterial count (bacterial count was carried out for each volume added, FIG. 1 shows the bacterial count for the volumes indicated in the Table for Bifidobacterium bifidum).

TABLE 2
HCl volume added per the different particles' ratio tested.

| | HCl (5M) added | HCl (5M) added |
|---|---|---|
| | (μl) to Lactobacillus | (μl) added to |
| MCC:CaCO₃ | crispatus and Lactobacillus | Bifidobacterium |
| Ratio | paracasei culture | bifidum culture |
| 75%:25% | 1000 | 1000 |
| 50%:50% | 2500 | 3000 |
| 25%:75% | 4000 | 4500 |

Resistance of Attached Bacterial Fraction to H₂O₂ Stress

The advantage of a composition comprising particle-attached bacteria prepared according to the invention in withstanding H₂O₂ stress conditions vs. planktonic bacterial composition and/or a bacterial composition comprising attached bacteria (96 w/p culturing) was examined.

The bacterial resistance to H₂O₂ was tested on several compositions: (i) a composition prepared according to an embodiment of the invention [culturing in the presence of MCC:CaCO₃ in a 1:1 (w/w) ratio]; (ii) a bacterial composition prepared on a 96 well-plate (marked as “96 w/p culturing”), and (iii) a planktonic bacterial composition (marked as “Planktonic”). Compositions (i) and (iii) were tested in a wet form (at the end of culturing) and following lyophilization. Composition (iii) prepared in a 96 w/p, was examined in a wet form. For measuring stress following lyophilization, the dried composition was reconstituted in PBS for 15 minutes at room temperature (at about 25° C.). Both wet and lyophilized forms were treated in the same manner as elaborated below.

Resistance experiments were carried out on Lactobacillus plantarum. The starting bacterial count in all tested conditions was similar.

### 96 w/p Culturing Procedure

As a comparison for the advantage of a composition according to an embodiment of the invention to withstand stress, culturing on 96 w/p was used, wherein bacterial growth is carried in an attached form.

A 96 w/p culture of Lactobacillus plantarum was prepared from an initial inoculum stored at −80° C. [the stock inoculum contained planktonic bacteria with 5% (v/v) DMSO solution].

Initially, 50 μl inoculum was mixed with 10 ml Lactobacillus plantarum medium (Yeast extract; Tween; L-cysteine; Sodium, Potassium, Manganese and Magnesium salts; and + (D)-Glucose) and incubated/cultured for 10 hours at a temperature of 37° C. under anaerobic conditions (Gas mix: N₂—90%, H₂—5%, CO₂—5%).

After the 10-hours incubation, OD₆₀₀ was measured, the bacterial population was diluted with Lactobacillus plantarum medium to an initial OD₆₀₀ of 0.01, and incubated for additional 12 hours under the same conditions while shaking at 100 RPM.

After the 12-hours incubation, OD₆₀₀ was measured, the bacterial population was diluted with Lactobacillus plantarum medium to an initial OD₆₀₀ of 0.01. The new bacterial stock was transferred to a 96 w/p in triplicates (100 μl in each well) and incubated for about 24 hours under the same conditions elaborated above under static conditions.

Following incubation, the spent solution was aspirated, and the culture was gently washed with PBS for removing the planktonic bacterial fraction (non-attached to the 96 w/p). The supernatant was aspirated from the well-plate. In the next step, the plate-attached bacterial population was exposed to increasing concentrations of H₂O₂ by adding a medium containing the H₂O₂ in the manner elaborated below.

### Culturing of Bacteria According to an Embodiment of the Invention in a Fermenter

Culturing was carried out in a 5 L fermenter containing 2 L of growth medium and in the presence of 400 g a particle mix [MCC and CaCO₃ in a 1:1 (w/w) ratio (200 g: 200 g)] in the manner elaborated below under: “Fermentation procedure”. The particles were pre-mixed with the medium prior to addition of the inoculum to the fermenter (the pH of the medium was adjusted to the culturing conditions-see below).

The medium used for culturing included: Yeast extract; Peptone; Tween; L-cysteine; Sodium, Potassium, Manganese and Magnesium salts; and + (D)-Glucose.

Oxidative stress, a well-accepted assay, was chosen as an exemplary stress condition to examine the advantage of a composition comprising particle-attached bacteria prepared according to the invention.

### Fermentation Procedure

A culture of Lactobacillus plantarum was prepared from an initial inoculum stored at −80° C. (the stock inoculum contained concentrated planktonic bacteria with glucose 50% solution in a ratio of 5:1 w/v).

A hundred (100) μl inoculum was mixed with 300 ml medium, and incubated for 10 hours under the following conditions: pH in the range ˜5.0-6.5; and a temperature of 37° C. under anaerobic conditions (Gas mix: N₂—90%, H₂—5%, CO₂—5%) without stirring.

Fifty (50) ml of the culture were inoculated into a fermenter containing 2 L media and 400 g particle mix. Fermentation/culturing was carried out for 15 hours, with constant stirring. Initial pH was 6.9 and the final pH was set to 5.94, temperature was set to 37° C.

Planktonic growth was carried out under corresponding conditions in the absence of particles.

As preparation for stress measurement of the composition according to the invention, following incubation, the particle-attached fraction was separated from the non-attached fraction (e.g., planktonic phase) by centrifugation (3 min at 48×g; at 21±2° C.), the supernatant was removed, followed by an additional PBS wash and centrifugation (5 minutes at 4,248×g; at 21±2° C.). The supernatant was removed, and the pellet was subjected to H₂O₂ stress.

As preparation for stress measurement of the planktonic culturing, the culture was centrifuged, the spent medium was removed, followed by an additional wash with PBS and an additional centrifugation (both centrifugations were carried for 5 minutes at 4,248×g; at 21±2° C.), and stress was applied thereafter.

H₂O₂ Stress

The bacteria were subjected to different concentrations of H₂O₂: 0.1, 0.2 or 0.5% for 1 minute. The different concentrations of H₂O₂ were prepared in a Lactobacillus plantarum culturing medium.

For stopping the H₂O₂ induced stress, 0.2% sodium thiosulfate in PBS was added to the culture.

For quantitatively assessing bacterial survival post-stress, bacteria grown in 96 w/p were detached from the surface of the well by vigorous pipetting; and bacteria grown in the presence of MCC:CaCO₃ were detached from the particles by vortex (at high speed for 1.5 minutes). The planktonic culture was also subjected to vortex. Total bacterial count, represented as colony forming units (CFU), was carried out using serial dilution in PBS and plating on MRS plate. The results are presented in percentages as compared to the control of the respective group (‘no addition of H₂O₂’ considered as 100%).

### Example 1

### Culturing of Bacterial Population in the Presence of MCC and CaCO₃ Particles

The following example examines the attachment level during culturing when using a particle mix of MCC and CaCO₃. For this purpose, culturing in the presence of the particle mix was compared to culturing in the presence of only MCC or only CaCO₃ particles. Planktonic culturing without particles was carried out in these experiments as reference.

Three different bacteria were cultured separately: Bifidobacterium bifidum, Lactobacillus paracasei, and Lactobacillus crispatus. The different bacteria were cultured as detailed above under “Culturing conditions and preparation of a bacterial composition” and “Experimental procedure” sections using different ratios of the particle mix, and pure MCC or pure CaCO₃ particles. Following culturing, the attachment level was tested and compared between the different tested groups.

FIG. 1 shows the bacterial count (CFU/ml) of Bifidobacterium bifidum grown without particles (planktonic growth form; marked as “Planktonic”); in the presence of particles for the combined particle-attached and non-attached bacterial fractions (marked as “Group 1”); only in the particle-attached bacterial fraction (marked as “Group 2”); and following partial elimination of the metal carbonate particles from the particle-attached bacterial fraction, i.e., from Group 2, by acid (marked as “Group 3”).

The results show that culturing of Bifidobacterium bifidum in the presence of a particle mix resulted in increased attachment as compared to growth in the presence of 100% MCC particles (compare in FIG. 1; Group 2 culturing in the presence of 100% MCC particles vs. culturing in the presence of both MCC and CaCO₃ yielding an increase from Log 7.92 CFU/ml to Log 8.68-8.87 CFU/ml).

The results also show (FIG. 1) that culturing of Bifidobacterium bifidum in the presence of a particle mix resulted in an increase in the total bacterial count—Log 9.35-9.49 CFU/ml (i.e., particle-attached and non-attached bacterial fractions; Group 1) as compared to the other bacterial growth forms-planktonic culturing without particles (Log 8.10 CFU/ml) or culturing in the presence of 100% MCC particles (Log 8.49 CFU/ml).

Culturing of Lactobacillus crispatus and Lactobacillus paracasei also corroborated the results demonstrating a trend of increase in the total bacterial count in the combined particle-attached and non-attached bacterial fractions and in the attachment level when culturing on a particle mix (using the concentrations elaborated above) vs. culturing on 100% MCC particles.

The results also demonstrate that culturing of various bacteria in the presence of 100% CaCO₃ particles, without MCC particles, resulted in a viscous culturing media which generally required an increase in the stirring speed in order to obtain a homogenous culturing media, thereby negatively affecting the level of bacterial attachment to the particles.

It can also be seen (FIG. 1) that adding acid to a composition comprising a CaCO₃-MCC particle mix had no significant effect on the bacterial count (compare the CFU/ml value of the “titrated particles”, Group 3, with the CFU/ml value of the “attached fraction”, Group 2). The obtained bacterial count following titration were even higher as compared to the bacterial count in the “attached fraction” of an MCC grown composition. The feasibility of eliminating the CaCO₃ particles with an acid while maintaining a high bacterial count was also corroborated with culturing of Lactobacillus crispatus and Lactobacillus paracasei (data not shown).

For Lactobacillus crispatus, Lactobacillus paracasei and Bifidobacterium bifidum, the pH obtained following addition of the indicated volumes of HCl was 4.0. Additional volumes of HCl, until complete dissolution of calcium carbonate were tested for Lactobacillus crispatus and Lactobacillus paracasei (as observed by CO₂ release from the solution), reaching a pH level of lower than 3.0. Such low PH levels reduced the bacterial viability by 14-45%. Advantageously, for obtaining optimal bacterial viability, the acid should be added in amount that the viability of the bacteria is substantially maintained.

### Example 2

### Increased Bacterial Attachment to a MCC-CaCO₃ Particle Mix

In the following experiment, the effect of the ratio between the amount of the particles and the volume of the culturing media on the level of bacterial attachment was examined.

Culturing was carried out in the presence of 50%-50% MCC-CaCO₃ particle mix and different ratios (up to 200 g particles per 1 L culturing medium) (culturing was carried out as elaborated above).

The results showed that a ratio between 60 g particle mix (wherein calcium carbonate constituted 50% (w/w) out of the total weight of the particles) per 1 L culturing medium and 200 g particle mix per 1 L culturing medium provided enhanced bacterial attachment to particles (data not presented).

A composition according to an embodiment of the invention can be prepared by contacting, incubating or culturing a population of at least one strain of bacteria in the presence of particles comprising: (i) a polysaccharide-based material, e.g., MCC; and (ii) a metal carbonate, e.g., CaCO₃, wherein the total weight of the particles with respect to the volume of the culturing solution can be in a range of between 60 g per 1 L to 250 g per 1 L, and wherein the metal carbonate can constitute between 15% and 85% w/w out of the total weight of the particles used, provided or added in the method.

Accordingly, for obtaining increased attachment, the ratio between the metal carbonate particles and the culturing solution used during contacting can advantageously be in a range of between 9 g per 1 L and 215 g per 1 L, taking into consideration that an advantageous metal carbonate content ranges between 15% and 85% w/w out of the total weight of the total particles.

In methods wherein the particles further comprise metal phosphate, e.g., DCP, the metal phosphate, and the metal carbonate can constitute together at least 15% w/w out of the total weight of the particles. All weight ratios are based on the dry weight of the particles.

Typically, culturing with metal carbonate at a concentration of higher than 85% (w/w out of the total weight of the particles), in the above specified particles: solution ratio, yields a viscous culturing medium that requires an increase in the stirring speed to obtain a homogenous culturing media, thereby negatively affecting bacterial attachment to the particles. Also, using metal carbonate in a concentration of lower than 15% (w/w out of the total weight of the particles), results in a lower bacterial attachment.

A composition can be manufactured wherein culturing is carried out in the presence of particles having a total weight with respect to the volume of the solution of lower than 60 g per 1 L. Under such conditions, the metal carbonate concentration can be between 15% and 99% (w/w) out of the total weight of the particles used in the method.

A composition prepared according to an embodiment of the invention can comprise both attached and non-attached bacterial fractions. Alternatively, the two bacterial fractions can be separated, e.g., by filtration and/or gravitational sedimentation, e.g., centrifugation as exemplified herein for carrying out CFU measurements on each separated bacterial fraction.

To examine the effect of consumable calcium on bacterial attachment to particles, different bacterial populations were cultured in the presence of MCC particles in different media compositions with or without water-soluble calcium salt (CaCl₂); 0.1%), and the effect of the presence of consumable calcium on the attachment level was examined.

The results showed that including calcium ion in the culturing media did not enhance bacterial attachment to the MCC particles (data not presented).

Taken together, the results exemplify the advantage of culturing in the presence of a particle mix according to the invention vs. culturing in the presence of pure particles of either MCC or CaCO₃ for achieving an increased bacterial attachment to particles and/or an increase in the total bacterial count of the composition. The results also demonstrated the feasibility of at least partially eliminating the particles during and/or after the culturing step, e.g., the CaCO₃ particles, from the bacterial composition while maintaining an increased bacterial count.

### Example 3

### Resistance of Different Bacterial Compositions to External Stress

The following example examines the advantage of a composition prepared according to an embodiment of the invention in withstanding stress conditions vs. planktonic bacterial composition and/or a bacterial composition comprising attached bacteria (96 w/p culturing). The bacterial compositions were tested in wet and lyophilized forms. Oxidative stress, a well-accepted assay, was chosen as an exemplary stress condition.

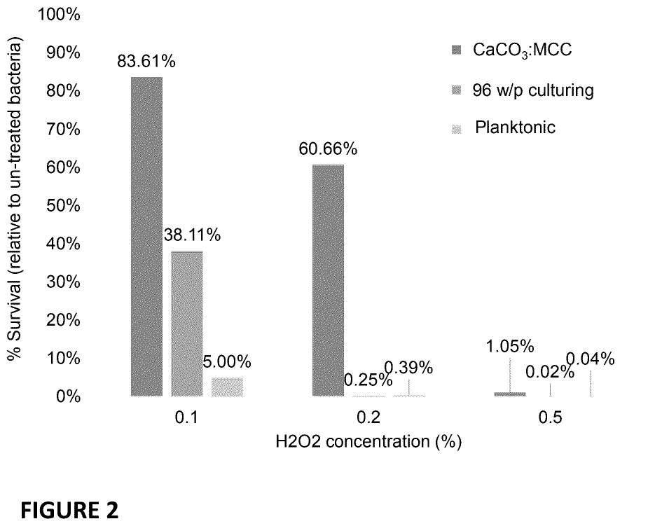

FIG. 2 shows the survival rate of a wet (at the end of culturing) Lactobacillus plantarum culture following exposure to increasing H₂O₂ concentrations. The resistance of the attached bacterial fraction prepared by different culturing methods were tested: (i) culturing according to an embodiment of the invention in the presence of CaCO₃:MCC particle mix (1:1 w/w ratio); and (ii) bacteria cultured on a 96 w/p. Planktonic growth form was used as a reference. Following the H₂O₂ exposure, the total bacterial count (CFU) was measured. The results are presented in percentages as compared to the control of the respective group (‘no addition of H₂O₂’ considered as 100%).

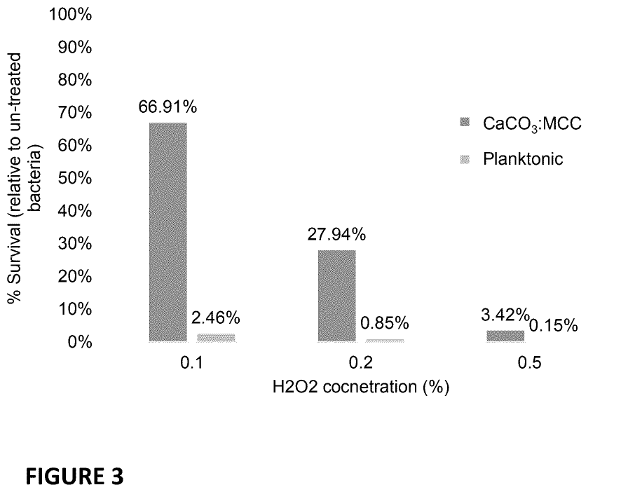

FIG. 3 shows the survival of lyophilized Lactobacillus plantarum culture following reconstitution and exposure to increasing H₂O₂ concentrations. The resistance was tested in culturing according to an embodiment of the invention in the presence of CaCO₃:MCC particle mix (1:1 w/w ratio); and in planktonic growth form. Following the H₂O₂ stress, the total bacterial count (CFU) was measured. The results are presented in percentages as compared to the control of the respective group (‘no addition of H₂O₂’ considered as 100%).

The results show a clear advantage for culturing in the presence of a particle mix according to the invention to withstand stress conditions as compared to other culturing methods, e.g., yielding a composition comprising an attached bacterial fraction and as compared to planktonic growth form.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A composition comprising a population of at least one strain of bacteria at least partially attached to particles, wherein said particles comprise: (i) a polysaccharide-based material; and (ii) a metal carbonate.

2. The composition of claim 1, wherein said particles are water-insoluble particles, and optionally wherein said metal carbonate is not being produced by said at least one strain of bacteria.

3. (canceled)

4. The composition of claim 1, having at least one characteristic selected from: (a) comprises said particles in a form of a composite comprising said polysaccharide-based material and said metal carbonate; and (b) comprises each of said polysaccharide-based material and said metal carbonate within a different particle.

5. The composition of claim 1, wherein said particles further comprise dicalcium phosphate (DCP).

6. The composition of claim 1, further comprising particles being devoid of polysaccharide-based material and metal carbonate.

7. The composition of claim 1, wherein said at least one strain of bacteria is attached to at least one of: a surface of said polysaccharide-based material; and a surface of said metal carbonate.

8. The composition of claim 1, wherein said polysaccharide-based material comprises a cellulose derivative.

9. The composition of claim 1, wherein said polysaccharide-based material comprises microcrystalline cellulose (MCC).

10. The composition of claim 1, wherein said metal carbonate comprises calcium carbonate.

11. The composition of claim 1, comprising said metal carbonate at a concentration of at most 85% (w/w) out of the total weight of said particles within said composition, based on the dry weight of said particles, and optionally wherein said composition being characterized by any one of: (i) comprising said metal carbonate at a concentration of at least 15% (w/w) out of the total weight of said particles within said composition, based on the dry weight of said particles; or (ii) further comprising metal phosphate comprising particles, and wherein said metal phosphate, and said metal carbonate constitute at least 15% (w/w) out of the total weight of said particles within said composition, based on the dry weight of said particles.

12. (canceled)

13. (canceled)

14. The composition of claim 1, wherein any one of: (i) said composition further comprising planktonic bacteria; (ii) said composition comprising two or more strains of bacteria; (iii) said composition being provided in solid form; (iv) said composition further comprising a pharmaceutically acceptable carrier or excipient; and (v) any combination of (i) to (iv).

15. (canceled)

16. (canceled)

17. (canceled)

18. A method for preparing a composition comprising a population of at least one strain of bacteria at least partially attached to particles, wherein said particles comprise: (i) a polysaccharide-based material; and (ii) a metal carbonate, the method comprising the steps of:
providing a population of at least one strain of bacteria;
providing particles comprising: (i) a polysaccharide-based material; and (ii) a metal carbonate;
contacting said population of at least one strain of bacteria with said particles, wherein said contacting is carried out in a solution; and
allowing said population of at least one strain of bacteria to at least partially attach to said particles;
thereby preparing said composition comprising said population of at least one strain of bacteria at least partially attached to said particles.

19. The method of claim 18, wherein said particles are water-insoluble particles.

20. The method of claim 18, wherein said contacting step comprises culturing said population of at least one strain of bacteria in a growth medium comprising said particles.

21. The method of claim 18, wherein during said contacting step, said particles have at least one characteristic selected from: (a) are in a form of a composite comprising said polysaccharide-based material and said metal carbonate; and (b) comprise each of said polysaccharide-based material and said metal carbonate within a different particle.

22. The method of claim 18, wherein said provided particles further comprise dicalcium phosphate (DCP).

23. The method of claim 18, wherein the weight of said metal carbonate with respect to the volume of said solution is in a range of between 9 g per 1 L and 215 g per 1 L, based on the dry weight of said particles.

24. The method of claim 1, wherein the total weight of said provided particles with respect to the volume of said solution is in a range of between 60 g per 1 L and 250 g per 1 L, based on the dry weight of said particles, and optionally wherein: (i) said provided particles comprise said metal carbonate at a concentration of at most 85% (w/w) out of the total weight of said particles, based on the dry weight of said particles; (ii) said provided particles are characterized by anyone of: (i) comprise said metal carbonate at a concentration of at least 15% (w/w) out of the total weight of said particles, based on the dry weight of said particles; or (ii) further comprise metal phosphate, and wherein said metal phosphate, and said metal carbonate constitute at least 15% out of the total weight of said particles, based on the dry weight of said particles; or (iii) both (i) and (ii).

25. (canceled)

26. (canceled)

27. The method of claim 18, wherein said metal carbonate is not being produced by said at least one strain of bacteria.

28. The method of claim 18, further comprising a step of eliminating said metal carbonate from said particles by adding an acid, CO₂, or both.
